(19) United States
(12) Patent Application Publication
(10) Pub. No.: US 2025/0044295 A1
(43) Pub. Date: Feb. 6, 2025

(54) BIOMARKERS FOR PREDICTING ELIGIBILITY FOR AN ANTI-ILT4 AND ANTI-PD-1 COMBINATION THERAPY

(71) Applicant: Merck Sharp & Dohme LLC, Rahway, NJ (US)

(72) Inventors: Rachel Allison Altura, Westerly, RI (US); Jared Lunceford, Washington, UT (US); Julia F. Markensohn, Westfield, NJ (US); Leah Suttner, Philadelphia, PA (US); Douglas C. Wilson, San Francisco, CA (US)

(73) Assignee: Merck Sharp & Dohme LLC, Rahway, NJ (US)

(21) Appl. No.: 18/720,011

(22) PCT Filed: Dec. 15, 2022
(86) PCT No.: PCT/US2022/052921

§ 371 Date: Jun. 14, 2024

Related U.S. Application Data
(60) Provisional application No. 63/290,145, filed on Dec. 16, 2021.

(51) Int. Cl. G01N 33/574; A61K 39/00; A61P 35/00; C07K 16/28

(52) U.S. Cl. CPC G01N 33/57492; A61P 35/00; C07K 16/2827; A61K 2039/507; G01N 2333/70532; G01N 2474/20

(57) ABSTRACT

Disclosed herein are biomarkers that correlate with responses to an anti-ILT4 and anti-PD-1 combination therapy. A biomarker that can differentiate responders from non-responders to this combination therapy can potentially be used to select human subjects who have a higher probability to benefit from such a combination therapy. In one embodiment, a combined positive score (CPS) for PD-L 1 expression in a tumor sample from a human subject is used as a biomarker to differentiate a responder from a non-responder to an anti-ILT4 and anti-PD-1 combination therapy. In another embodiment, a T-cell-inflamed gene expression profile (Tcell_{inf}GEP) score is used as a biomarker to differentiate a responder from a non-responder to an anti-ILT4 and anti-PD-1 combination therapy.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Jul. 15, 2022, is named 25349_WO_PCT_SL.XML and is 10.0 KB bytes in size.

### BACKGROUND OF THE INVENTION

The development of immune checkpoint inhibitors, including monoclonal antibodies against programmed death 1 (PD-1) or its ligand, PD-L1, has improved patient outcomes in those with advanced malignancies; however, many patients do not respond to these therapies or they acquire resistance to them (Pitt J M, Vétizou M, Daillére R, Roberti M P, Yamazaki T, Routy B, et al. Resistance mechanisms to immune-checkpoint blockade in cancer: tumor-intrinsic and extrinsic factors. Immunity 2016; 44:1255-69). Combining immunotherapies that target distinct mechanisms of immunosuppression may improve outcome or overcome resistance (Pitt J M, Vétizou M, Daillére R, Roberti M P, Yamazaki T, Routy B, et al. Resistance mechanisms to immune-checkpoint blockade in cancer: tumor-intrinsic and -extrinsic factors. Immunity 2016; 44:1255-69; Rouas-Freiss N, LeMaoult J, Verine J, Tronik-Le Roux D, Culine S, Hennequin C, et al. Intratumor heterogeneity of immune checkpoints in primary renal cell cancer: Focus on HLA-G/ILT2/ILT4. Oncoimmunology 2017;6:e1342023). In addition to the PD-1/PD-L1 axis, which suppresses antitumor effector T-cell responses (Hirano F, Kaneko K, Tamura H, Dong H, Wang S, Ichikawa M, et al. Blockade of B7-H1 and PD-1 by monoclonal antibodies potentiates cancer therapeutic immunity. Cancer Res 2005; 65:1089-96; Francisco L M, Salinas V H, Brown K E, Vanguri V K, Freeman G J, Kuchroo V K, Sharp A H. PD-L1 regulates the development, maintenance, and function of induced regulatory T cells. J Exp Med 2009; 206:3015-29), myeloid-derived suppressor cells (MDSCs) represent another major axis of immunosuppression (Fleming V, Hu X, Weber R, Nagibin V, Groth C, Altevogt P, et al. Targeting myeloid-derived suppressor cells to bypass tumor-induced immunosuppression. Front Immunol 2018; 9:398) and are associated with poor prognosis in patients with cancer (Ai L, Mu S, Wang Y, Wang H, Cai L, Li W. Prognostic role of myeloid-derived suppressor cells in cancers: a systematic review and meta-analysis. BMC Cancer 2018; 18:1220). MDSCs dampen T-cell activation, proliferation, and effector responses through numerous mechanisms including, but not limited to, cytokine production, cell surface receptor signaling, reactive oxygen species production, nutrient deprivation, and regulatory T-cell recruitment (Adah D, Hussain M, Qin L, Qin L, Zhang J, Chen X. Implications of MDSCs-targeting in lung cancer chemo-immunotherapeutics. Pharmacol Res 2016; 110:25-34). Given that MDSCs are a common constituent of the tumor microenvironment and because of their diverse arsenal of suppressive mechanisms, MDSCs are considered a prime target for therapeutic intervention, especially in combination with T-cell targeted immunotherapy. Several novel therapeutics targeting one or more of these mechanisms have recently been explored in early clinical trials including molecules targeting colony stimulating factor receptor 1 (CSFR1), C-X-C motif chemokine receptors 1/2 (CXCR1/2), adenosine receptors, and class 1 histone deacetylases (HDACs) (De Cicco, et al., Front. Immunol. 2020).

Immunoglobulin-like transcript 4 (ILT4) receptor, otherwise known as leukocyte immunoglobulin-like receptor B2 (LILRB2), is an immunosuppressive member of the immunoglobulin-like transcript family that is commonly expressed by many myeloid lineages including monocytes, macrophages, granulocytes, and dendritic cells (Colonna M, Navarro F, Bellón T, Llano M, Garcia P, Samaridis J, et al. A common inhibitory receptor for major histocompatibility complex class I molecules on human lymphoid and myelomonocytic cells. The Journal of experimental medicine 1997; 186:1809-18; Colonna M, Samaridis J, Cella M, Angman L, Allen R L, O'Callaghan C A, et al. Human myelomonocytic cells express an inhibitory receptor for classical and nonclassical MHC class I molecules. J Immunol 1998; 160:3096-100; Fanger N A, Cosman D, Peterson L, Braddy S C, Maliszewski C R, Borges L. The MHC class I binding proteins LIR-1 and LIR-2 inhibit Fc receptor-mediated signaling in monocytes. Eur J Immunol 1998; 28:3423-34; Gao A, Sun Y, Peng G. ILT4 functions as a potential checkpoint molecule for tumor immunotherapy. Biochim Biophys Acta Rev Cancer 2018; 1869:278-85). In the tumor microenvironment, ILT4 is expressed by cells with a phenotype associated with monocytic MDSCs (mMDSCs) and granulocytic MDSCs (gMDSCs) (Kostlin N, Ostermeir A L, Spring B, Schwarz J, Marmé A, Walter C B, et al. HLA-G promotes myeloid-derived suppressor cell accumulation and suppressive activity during human pregnancy through engagement of the receptor ILT4. Eur J Immunol 2017; 47:374-84). The prototypical ligand for ILT4 is human leukocyte antigen G (HLA-G), a nonclassical major histocompatibility complex class I molecule expressed by a wide range of tumors, and is correlated with poor prognosis (Colonna M, Samaridis J, Cella M, Angman L, Allen R L, O'Callaghan C A, et al. Human myelomonocytic cells express an inhibitory receptor for classical and nonclassical MHC class I molecules. J Immunol 1998; 160:3096-100; Cai Z, Wang L, Han Y, Gao W, Wei X, Gong R, et al. Immunoglobulin-like transcript 4 and human leukocyte antigen-G interaction promotes the progression of human colorectal cancer. Int J Oncol 2019; 54:1943-54; Li Q, Li J, Wang S, Wang J, Chen X, Zhou D, et al. Overexpressed immunoglobulin-like transcript (ILT) 4 in lung adenocarcinoma is correlated with immunosuppressive T cell subset infiltration and poor patient outcomes. Biomark Res 2020; 8:11). Several other ILT4 ligands, including classical human leukocyte antigens (HLAs) and angiopoietin-like proteins (ANGPTLs), also may be relevant in driving ILT4-mediated immunosuppression in the tumor microenvironment (Gao A, Sun Y, Peng G. ILT4 functions as a potential checkpoint molecule for tumor immunotherapy. Biochim Biophys Acta Rev Cancer 2018; 1869:278-85). Emerging data show that ILT4 antagonism in tumor-associated macrophages (TAMs) induces a more proinflammatory state, one that is marked by a shift from the M2-phenotype toward an M1-phenotype (Gao A, Sun Y, Peng G. ILT4 functions as a potential checkpoint molecule for tumor immunotherapy. Biochim Biophys Acta Rev Cancer 2018; 1869:278-85; Chen H M, van der Touw W, Wang Y S, Kang K, Mai S, Zhang J, et al. Blocking immunoinhibitory receptor LILRB2 reprograms tumor-associated myeloid cells and promotes antitumor immunity. J Clin Invest 2018; 128:5647-62). Reprogramming TAMs/MDSCs to a proinflammatory state may be more advantageous than targeting these cells for depletion because the resultant proinflammatory response from ILT4-inhibition may better stimulate the anti-tumor T cell response, especially in combination with PD-1/L1 blockade (Sharma et al., Immunity, Vol. 48, Issue 1, 91-106, 2018).

An anti-ILT4 antibody comprising a light chain sequence of SEQ ID No. 5 and a heavy chain sequence of SEQ ID No. 10, which is a fully human monoclonal antibody of the immunoglobulin G4 subclass that specifically binds to ILT4 and blocks its interaction with HLA-G and other ligands has been described (FIG. 1) (Zuniga L, Joyce-Shaikh B, Wilson D, Cherwinski H, Chen Y, Jeff G, et al. Preclinical characterization of a first-in-class ILT4 antagonist, MK-4830. J ImmunoTher Cancer 2018; 6:115. P532). Preclinical data demonstrated that this antibody binds to monocytes and granulocytes in the peripheral blood of healthy volunteers and patients with cancer. In a humanized mouse model of cancer, treatment with this antibody induced significant inhibition of tumor growth (Zuniga L, Joyce-Shaikh B, Wilson D, Cherwinski H, Chen Y, Jeff G, et al. Preclinical characterization of a first-in-class ILT4 antagonist, MK-4830. J ImmunoTher Cancer 2018; 6:115. P532).

Previous studies have established several tumor-associated biomarkers, including programmed death ligand 1 (PD-L1), tumor mutational burden (TMB), and microsatellite instability (MSI), that associate with a response to the anti-PD-1 antibody pembrolizumab, leading to a more comprehensive and selective treatment paradigm. An interferon-gamma (IFN-γ)-related, 18-gene, T-cell-inflamed gene expression profile (Tcell_{inf}GEP) signature has also been shown to be positively associated with a response to pembrolizumab monotherapy in several tumor types (Ayers M, Lunceford J, Nebozhyn M, Murphy E, Loboda A, Kaufman D R, et al. IFN-gamma-related mRNA profile predicts clinical response to PD-1 blockade. J Clin Invest 2017; 127:2930-40.).

Despite the availability of biomarkers that can assist with selecting monotherapy treatments involving PD-1, there is an unmet need for biomarkers that can help with predicting eligibility for an anti-ILT4 and anti-PD-1 combination therapy.

### SUMMARY OF THE INVENTION

Disclosed herein are biomarkers that correlate with responses to an anti-ILT4 and anti-PD-1 combination therapy. A biomarker that can differentiate responders from non-responders to this combination therapy can potentially be used to select human subjects who have a higher probability to benefit from such a combination therapy.

In one embodiment, a PD-L1 combined positive score (CPS) is used as a biomarker to differentiate a responder from a non-responder to an anti-ILT4 and anti-PD-1 combination therapy.

In one embodiment, a human subject with a PD-L1 CPS value of equal to or greater than 5; or more specifically, 6; 7; 8; 9; or 10, is a suitable subject for an anti-ILT4 and anti-PD-1 combination therapy. In one embodiment, the anti-ILT4 and anti-PD-1 combination therapy is a combination therapy of an anti-ILT4 antibody comprising a light chain sequence of SEQ ID No. 5 and a heavy chain sequence of SEQ ID No. 10, hereinafter referred to as “Antibody A”, and pembrolizumab.

In one embodiment, a human subject with a PD-L1 CPS value of equal to or greater than 5 is a suitable subject for Antibody A and pembrolizumab combination therapy.

In one embodiment, a T-cell-inflamed gene expression profile (Tcell_{inf}GEP) score is used as a biomarker to differentiate a responder from a non-responder to an anti-ILT4 and anti-PD-1 combination therapy. The distribution of the Tcell_{inf}GEP score is higher in responders than in non-responders.

In one embodiment, a human subject with a Tcell_{inf}GEP score of equal to or greater than −0.6; or more specifically, −0.5; −0.4; or −0.3, is a suitable subject for an anti-ILT4 and anti-PD-1 combination therapy. In one embodiment, the anti-ILT4 and anti-PD-1 combination therapy is a combination therapy of Antibody A and pembrolizumab.

In one embodiment, a subject with a Tcell_{inf}GEP score of equal to or greater than −0.5 is a suitable subject for Antibody A and pembrolizumab combination therapy.

### BRIEF DESCRIPTION OF THE FIGURES

FIG. 1 provides a cartoon showing an anti-ILT4 and anti-PD-1 combination therapy.

FIGS. 2A-2D provides graphs of target lesion change over time (RECIST v1.1) in patients who received Antibody A monotherapy (FIG. 2A) or Antibody A in combination with pembrolizumab (FIG. 2B); and best percentage change from baseline in target lesion based on investigator assessment (RECIST v1.1) in patients who received Antibody A monotherapy (FIG. 2C) or Antibody A in combination with pembrolizumab (FIG. 2D).

FIGS. 3A-3C provides association between response and PD-L1 status (FIG. A), TMB status (FIG. 3B), or Tcell_{inf}GEP score (FIG. 3C). AUROC, area under the receiver operating characteristic; BOR, best overall response; CI, confidence interval; CPS, combined positive score; CR, complete response; NR, non-responder; ORR, objective response rate; PD, progressive disease; PD-L1, programmed death ligand 1; PR, partial response; R, responder; ROC, receiver operating characteristic; SD, stable disease; Tcell_{inf}GEP, T-cell-inflamed gene expression profile; TMB, tumor mutational burden.

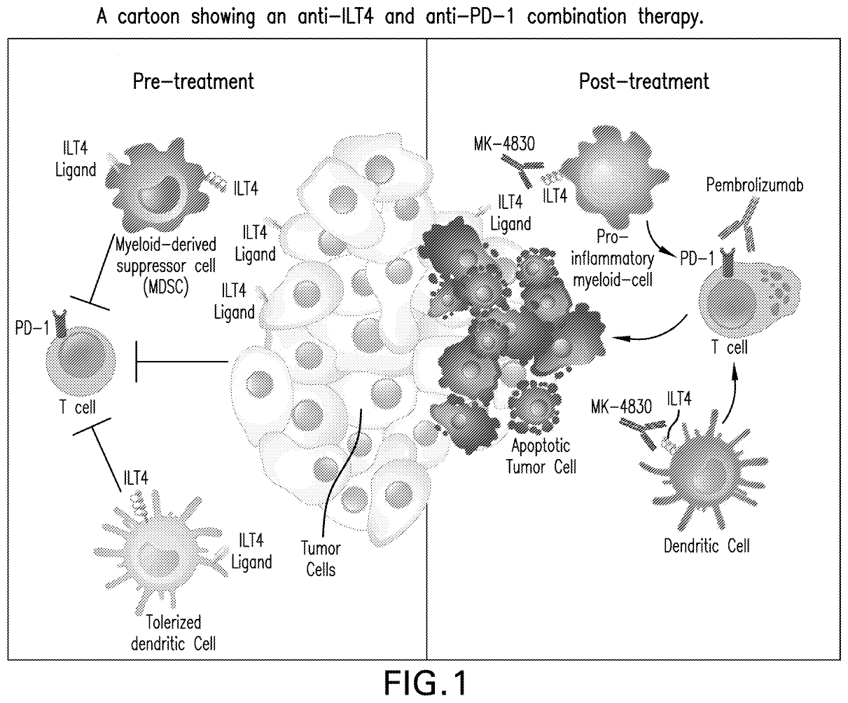

### DETAILED DESCRIPTION OF THE INVENTION

Disclosed herein are biomarkers and methods for determining the eligibility of a human subject having a malignancy for treatment with an anti-ILT4 and anti-PD-1 combination therapy. As used herein, an “anti-ILT4 and anti-PD-1 combination therapy” refers to a method of treatment that comprises administration of an anti-ILT4 therapy (e.g., an anti-ILT4 antibody or antigen binding fragment thereof) and an anti-PD-1 therapy (e.g., an anti-PD-1 antibody or antigen binding fragment thereof). In some embodiments, an anti-ILT4 and anti-PD-1 combination therapy comprises administration of an anti-ILT4 antibody or antigen binding fragment thereof and an anti-PD-1 antibody or antigen binding fragment thereof simultaneously (e.g. a co-formulation). In alternate embodiments, an anti-ILT4 and anti-PD-1 combination therapy comprises administration of an anti-ILT4 antibody or antigen binding fragment thereof and an anti-PD-1 antibody or antigen binding fragment thereof concurrently, sequentially, or for overlapping periods of time.

In one embodiment, a T-cell-inflamed gene expression profile (Tcell_{inf}GEP) score is used as a biomarker to differentiate a responder from a non-responder to an anti-ILT4 and anti-PD-1 combination therapy. The distribution of the Tcell_{inf}GEP score is higher in responders than in non-responders.

In one embodiment, a human subject with a Tcell_{inf}GEP score of equal to or greater than −0.6; or more specifically, −0.5; −0.4; or −0.3, is a suitable subject for an anti-ILT4 and anti-PD-1 combination therapy. In one embodiment, the anti-ILT4 and anti-PD-1 combination therapy is a combination therapy of Antibody A and pembrolizumab.

In one embodiment, a human subject with a Tcell_{inf}GEP score of equal to or greater than −0.5 is a suitable subject for an anti-ILT4 and anti-PD-1 combination therapy.

In one embodiment, a human subject with a Tcell_{inf}GEP score of equal to or greater than −0.5 is a suitable subject for an anti-ILT4 antibody and pembrolizumab combination therapy. In one embodiment, the anti-ILT4 antibody is Antibody A. In one embodiment, the anti-ILT4 antibody comprises a VL sequence of SEQ ID No. 4 and a VH sequence of SEQ ID No. 9. In one embodiment, the anti-ILT4 antibody comprises a VL-CDR1 of SEQ ID No. 1, a VL-CDR2 of SEQ ID No. 2, and a VL-CDR3 of SEQ ID No. 3; and a VH-CDR1 of SEQ ID No. 6, a VH-CDR2 of SEQ ID No. 7, and a VH-CDR3 of SEQ ID No. 8.

In one embodiment, a Combined Positive Score (CPS) for PD-L1 expression in a tumor sample from a human subject is used as a biomarker to differentiate a responder from a non-responder to an anti-ILT4 and anti-PD-1 combination therapy. The disclosed CPS biomarker can decrease the workload for pathologists in the clinic, is easier to apply, produces higher inter-observer and intra-observer concordances, and identifies subjects eligible for the combination therapy who would otherwise be missed using other scoring methods focusing solely on biomarker expression on tumor cells.

In one embodiment, a PD-L1 CPS is determined using baseline (archival) tumor samples from a human cancer subject.

In one embodiment, a PD-L1 CPS is used as a biomarker to differentiate a responder from a non-responder to an anti-ILT4 and anti-PD-1 combination therapy, wherein a response is defined by objective response rate (ORR). In one embodiment, a response is defined by disease control rate (DCR). In another embodiment, a response is defined by ORR and DCR.

In one embodiment, a PD-L1 CPS is used as a biomarker to identify a human subject being treated with an anti-ITL4 antibody who would respond to an anti-ILT4 and anti-PD-1 combination therapy.

In one embodiment, a human subject with a higher PD-L1 CPS value has a higher probability of responding to an anti-ILT4 and anti-PD-1 combination therapy. In one embodiment, a subject with a PD-L1 CPS value of 1, 2, 3, 4, 5, 6, 7, 8, 9, 10, 15, 20, 25, 30, 40, or 50 is a suitable subject for an anti-ILT4 and anti-PD-1 combination therapy.

In one embodiment, a human subject with a PD-L1 CPS value of 1, 2, 3, 4, 5, 6, 7, 8, 9, 10, 15, 20, 25, 30, 40, or 50 is a suitable subject for an anti-ILT4 antibody and pembrolizumab combination therapy. In one embodiment, the anti-ILT4 antibody comprises a VL-CDR1 of SEQ ID No. 1, a VL-CDR2 of SEQ ID No. 2, and a VL-CDR3 of SEQ ID No. 3; and a VH-CDR1 of SEQ ID No. 6, a VH-CDR2 of SEQ ID No. 7, and a VH-CDR3 of SEQ ID No. 8. In one embodiment, the anti-ILT4 antibody is Antibody A. In one embodiment, the anti-ILT4 antibody comprises a VL sequence of SEQ ID No. 4 and a VH sequence of SEQ ID No. 9. In one embodiment, the anti-ILT4 antibody comprises a VL-CDR1 of SEQ ID No. 1, a VL-CDR2 of SEQ ID No. 2, and a VL-CDR3 of SEQ ID No. 3; and a VH-CDR1 of SEQ ID No. 6, a VH-CDR2 of SEQ ID No. 7, and a VH-CDR3 of SEQ ID No. 8.

A human subject is eligible for anti-ILT4 and anti-PD-1 combination therapy if the PD-L1 CPS value determined from a specimen of the subject is equal to or greater than a specified threshold value. For example, a suitable human subject with a PD-L1 CPS value of 5 means that a subject with a CPS value of equal to or greater than 5 is a suitable subject for an anti-ILT4 and anti-PD-1 combination therapy.

A PD-L1 CPS value is determined using a method comprising determining the number of viable PD-L1 positive tumor cells, the number of viable PD-L1 negative tumor cells, and the number of viable PD-L1 positive mononuclear inflammatory cells (MIC) in a tumor tissue sample from a human subject having a malignancy; and calculating a combined positive score (CPS) for the tumor tissue sample using the formula:

$$CPS = \frac{PD - L 1 positive tumor cells + PD - L 1 positive MIC}{PD - L 1 positive tumor cells + PD - L 1 negative tumor cells} \times 100 ;$$

wherein the subject is eligible for treatment with an anti-ILT4 and anti-PD-1 combination therapy when the CPS is above a threshold value.

Although the score can be calculated as greater than 100, the maximum CPS is defined as 100.

A human subject is eligible for anti-ILT4 and anti-PD-1 combination therapy if the CPS value determined from a specimen of the subject is equal to or greater than a threshold value. Thresholds can be determined by a receiver operating characteristic (ROC) curve for best responder/cut off (threshold) correlation. Threshold (or cut offs/cut points) can thus be any applicable CPS value, including but not limited to 1, 2, 3, 4, 5, 6, 7, 8, 9, 10, 15, 20, 25, 30, 35, 40, 45, and 50, and is specific to a particular combination therapy used.

In one embodiment, a method for determining the eligibility of a human subject having a malignancy for treatment with an anti-ILT4 and anti-PD-1 combination therapy comprises: determining the number of viable PD-L1 positive tumor cells, the number of viable PD-L1 negative tumor cells, and the number of viable PD-L1 positive mononuclear inflammatory cells (MIC) in a tumor tissue sample from the subject having a malignancy; and calculating a combined positive score (CPS) for the tumor tissue sample using the formula:

$$CPS = \frac{PD - L 1 positive tumor cells + PD - L 1 positive MIC}{PD - L 1 positive tumor cells + PD - L 1 negative tumor cells} \times 100 ;$$

wherein the subject is eligible for treatment with the anti-ILT4 and anti-PD-1 combination therapy when the CPS is equal to or greater than a threshold value.
   In one embodiment of the above method, the threshold value is selected from 5, 6, 7, 8 and 9.

In one embodiment of the above method, the threshold value is selected from 10, 15, 20, 25, 30, 35 and 40.

In one embodiment of the above method, the threshold value is 5. In one embodiment of the above method, the threshold value is 6. In one embodiment of the above method, the threshold value is 7. In one embodiment of the above method, the threshold value is 8. In one embodiment of the above method, the threshold value is 9. In one embodiment of the above method, the threshold value is 10. In one embodiment of the above method, the threshold value is 15. In one embodiment of the above method, the threshold value is 20. In one embodiment of the above method, the threshold value is 25. In one embodiment of the above method, the threshold value is 30. In one embodiment of the above method, the threshold value is 35. In one embodiment of the above method, the threshold value is 40.

In one embodiment of the above method, the tumor tissue sample is a tissue section of a tumor biopsy.

In one embodiment of the above method, PD-L1 is detected by immunohistochemistry (IHC) staining.

In one embodiment of the above method, the tumor tissue section is a formalin fixed and embedded in paraffin wax (FFPE) tumor tissue section.

In one embodiment of the above method, the tissue section is stained.

In one embodiment of the above method, the stain comprises a hematoxylin and eosin (H&E) stain.

In one embodiment of the above method, the viable PD-L1 positive tumor cells, the number of viable PD-L1 negative tumor cells, and the number of viable PD-L1 positive MIC are counted in the tumor nests and the adjacent supporting stroma of the tumor tissue sample.

In one embodiment of the above method, the number of viable PD-L1 positive tumor cells, the number of viable PD-L1 negative tumor cells, and the number of viable PD-L1 positive MIC in the tumor tissue sample are determining by flow cytometry.

In one embodiment of the above method, the method further comprises, prior to the determining step, contacting the tumor tissue sample with an anti-ILT4 antibody or a binding fragment thereof and an anti-PD-1 antibody or a binding fragment thereof.

In one embodiment of the above method, the anti-ILT4 antibody or a binding fragment thereof is Antibody A.

In one embodiment of the above method, the anti-PD-1 antibody or a binding fragment thereof is selected from the group consisting of: nivolumab, pembrolizumab, dostarlimab, cemiplimab, BMS-936559, MPDL3280A, and MEDI4736.

In one embodiment of the above method, the anti-PD-1 antibody or a binding fragment thereof is pembrolizumab.

In one embodiment of the above method, the anti-ILT4 antibody or a binding fragment thereof is Antibody A; and the anti-PD-1 antibody or a binding fragment thereof is pembrolizumab.

In one embodiment of the above method, the malignancy is selected from the group consisting of: gastric cancer, head and neck cancer, renal cell carcinoma, urothelial carcinoma, ovarian carcinoma, myeloma, melanoma, lung cancer, squamous cell carcinoma, classical Hodgkin lymphoma, breast cancer, triple negative breast cancer, hormone receptor positive (ER and/or PR) and Her2 positive breast cancer, small cell lung cancer, salivary gland carcinoma, vulvar carcinoma, thyroid carcinoma, anal canal carcinoma, biliary carcinoma, mesothelioma, cervical carcinoma, and neuroendocrine carcinoma.

As disclosed herein, when a PD-L1 CPS threshold value is 5, the specimen is considered PD-L1 positive if CPS≥5 (i.e., equal to or greater than the threshold value of 5). PD-L1 positive cells are viable cells (tumor or mononuclear inflammatory cells) that exhibit membrane staining at any intensity over background. In one embodiment, the threshold value is 2. In one embodiment, the threshold value is 3. In one embodiment, the threshold value is 4. In one embodiment, the threshold value is 6. In one embodiment, the threshold value 7. In one embodiment, the threshold value is 8. In one embodiment, the threshold value is 9. In one embodiment, the threshold value is 10. In one embodiment, the threshold value is 15. In one embodiment, the threshold value is 20. In one embodiment, the threshold value is 25. In one embodiment, the threshold value is 30. In one embodiment, the threshold value is 35. In one embodiment, the threshold value is 40.

In one embodiment there is provided a method for treating a human subject having a malignancy comprises the steps of:
determining the number of viable PD-L1 positive tumor cells, the number of viable PD-L1 negative tumor cells, and the number of viable PD-L1 positive mononuclear inflammatory cells (MIC) in a tumor tissue sample from the subject; and
calculating a combined positive score (CPS) for the tumor tissue sample using the formula:

$$CPS = \frac{PD - L 1 positive tumor cells + PD - L 1 positive MIC}{PD - L 1 positive tumor cells + PD - L 1 negative tumor cells} \times 100 ;$$

wherein the subject is treated with the anti-ILT4 and anti-PD-1 combination therapy when the CPS is equal to or greater than a threshold value.

In one embodiment of the above method, the threshold value is selected from 5, 6, 7, 8 and 9.

In one embodiment of the above method, the threshold value is 5.

In one embodiment of the above method, the tumor tissue sample is a tissue section of a tumor biopsy.

In one embodiment of the above method, PD-L1 is detected by immunohistochemistry (IHC) staining.

In one embodiment of the above method, the tumor tissue section is a formalin fixed and embedded in paraffin wax (FFPE) tumor tissue section.

In one embodiment of the above method, the tissue section is stained.

In one embodiment of the above method, the stain comprises a hematoxylin and eosin (H&E) stain.

In one embodiment of the above method, the viable PD-L1 positive tumor cells, the number of viable PD-L1 negative tumor cells, and the number of viable PD-L1 positive MIC are counted in the tumor nests and the adjacent supporting stroma of the tumor tissue sample.

In one embodiment of the above method, the number of viable PD-L1 positive tumor cells, the number of viable PD-L1 negative tumor cells, and the number of viable PD-L1 positive MIC in the tumor tissue sample are determining by flow cytometry.

In one embodiment of the above method, the method further comprises, prior to the determining step, contacting the tumor tissue sample with an anti-ILT4 antibody or a binding fragment thereof and an anti-PD-1 antibody or a binding fragment thereof.

In one embodiment of the above method, the anti-ILT4 antibody or a binding fragment thereof comprises a VL-CDR1 of SEQ ID No. 1, a VL-CDR2 of SEQ ID No. 2, and a VL-CDR3 of SEQ ID No. 3; and a VH-CDR1 of SEQ ID No. 6, a VH-CDR2 of SEQ ID No. 7, and a VH-CDR3 of SEQ ID No. 8.

In one embodiment of the above method, the anti-ILT4 antibody or a binding fragment thereof is Antibody A which comprises a light chain sequence of SEQ ID No. 5 and a heavy chain sequence of SEQ ID No. 10.

In one embodiment of the above method, the anti-PD-1 antibody or a binding fragment thereof is selected from the group consisting of: nivolumab, pembrolizumab, dostarlimab, cemiplimab, BMS-936559, MPDL3280A, and MEDI4736.

In one embodiment of the above method, the anti-ILT4 antibody or a binding fragment thereof is Antibody A which comprises a light chain sequence of SEQ ID No. 5 and a heavy chain sequence of SEQ ID No. 10; and wherein the anti-PD-1 antibody or a binding fragment thereof is pembrolizumab.

In one embodiment of the above method, the malignancy is selected from the group consisting of: gastric cancer, head and neck cancer, renal cell carcinoma, urothelial carcinoma, ovarian carcinoma, myeloma, melanoma, lung cancer, squamous cell carcinoma, classical Hodgkin lymphoma, breast cancer, triple negative breast cancer, hormone receptor positive (ER and/or PR) and Her2 positive breast cancer, small cell lung cancer, salivary gland carcinoma, vulvar carcinoma, thyroid carcinoma, anal canal carcinoma, biliary carcinoma, mesothelioma, cervical carcinoma, and neuroendocrine carcinoma.

In one embodiment, a method for treating a human subject having a malignancy comprises the steps of:
treating the subject with an anti-ILT4 antibody or a binding fragment thereof,
determining the number of viable PD-L1 positive tumor cells, the number of viable PD-L1 negative tumor cells, and the number of viable PD-L1 positive mononuclear inflammatory cells (MIC) in a tumor tissue sample from the subject who has been treated with the anti-ILT4 antibody or a binding fragment thereof, and
calculating a combined positive score (CPS) for the tumor tissue sample using the formula:

$$CPS = \frac{PD - L 1 positive tumor cells + PD - L 1 positive MIC}{PD - L 1 positive tumor cells + PD - L 1 negative tumor cells} \times 100 ;$$

wherein the subject is further treated with the anti-ILT4 and anti-PD-1 combination therapy when the CPS is equal to or greater than a threshold value.

In one embodiment of the above method, the threshold value is selected from 5, 6, 7, 8 and 9.

In one embodiment of the above method, the threshold value is 5.

In one embodiment of the above method, the tumor tissue sample is a tissue section of a tumor biopsy.

In one embodiment of the above method, PD-L1 is detected by immunohistochemistry (IHC) staining.

In one embodiment of the above method, the tumor tissue section is a formalin fixed and embedded in paraffin wax (FFPE) tumor tissue section.

In one embodiment of the above method, the tissue section is stained.

In one embodiment of the above method, the stain comprises a hematoxylin and eosin (H&E) stain.

In one embodiment of the above method, the viable PD-L1 positive tumor cells, the number of viable PD-L1 negative tumor cells, and the number of viable PD-L1 positive MIC are counted in the tumor nests and the adjacent supporting stroma of the tumor tissue sample.

In one embodiment of the above method, the number of viable PD-L1 positive tumor cells, the number of viable PD-L1 negative tumor cells, and the number of viable PD-L1 positive MIC in the tumor tissue sample are determining by flow cytometry.

In one embodiment of the above method, the method further comprises, prior to the determining step, contacting the tumor tissue sample with an anti-ILT4 antibody or a binding fragment thereof and an anti-PD-1 antibody or a binding fragment thereof.

In one embodiment of the above method, the anti-ILT4 antibody or a binding fragment thereof comprises a VL-CDR1 of SEQ ID No. 1, a VL-CDR2 of SEQ ID No. 2, and a VL-CDR3 of SEQ ID No. 3; and a VH-CDR1 of SEQ ID No. 6, a VH-CDR2 of SEQ ID No. 7, and a VH-CDR3 of SEQ ID No. 8.

In one embodiment of the above method, the anti-ILT4 antibody or a binding fragment thereof is Antibody A which comprises a light chain sequence of SEQ ID No. 5 and a heavy chain sequence of SEQ ID No. 10.

In one embodiment of the above method, the anti-PD-1 antibody or a binding fragment thereof is selected from the group consisting of: nivolumab, pembrolizumab, dostarlimab, cemiplimab, BMS-936559, MPDL3280A, and MEDI4736.

In one embodiment of the above method, the anti-ILT4 antibody or a binding fragment thereof is Antibody A which comprises a light chain sequence of SEQ ID No. 5 and a heavy chain sequence of SEQ ID No. 10; and wherein the anti-PD-1 antibody or a binding fragment thereof is pembrolizumab.

In one embodiment of the above method, the malignancy is selected from the group consisting of: gastric cancer, head and neck cancer, renal cell carcinoma, urothelial carcinoma, ovarian carcinoma, myeloma, melanoma, lung cancer, squamous cell carcinoma, classical Hodgkin lymphoma, breast cancer, triple negative breast cancer, hormone receptor positive (ER and/or PR) and Her2 positive breast cancer, small cell lung cancer, salivary gland carcinoma, vulvar carcinoma, thyroid carcinoma, anal canal carcinoma, biliary carcinoma, mesothelioma, cervical carcinoma, and neuroendocrine carcinoma.

In any of the embodiments above, the human subject may be treated with an anti-ILT4 antibody, including Antibody A, prior to the determining of the suitability of treatment of the subject with anti-ILT4 and anti-PD-1 combination therapy.

In one embodiment, the tissue sample (or specimen) must contain a minimum number of viable cells for evaluation, e.g., at least 50 viable tumor cells, at least 100 viable tumor cells, at least 150 viable tumor cells, at least 250 viable tumor cells, etc. By viable cells is meant that the cells were viable at the time they were harvested from the subject, and not necessarily viable at the time of staining. For example, where tumor biopsy sample slides are analyzed, the cells are considered viable cells if they are deemed, upon observation and analysis, to have been viable at the time the tumor biopsy was harvested. If the specimen contains the minimum number of viable cells, the specimen is evaluable using the CPS method, e.g., by calculating an eligibility score.

Detecting PD-L1 positive cells in a tumor sample can be done in any convenient manner. In certain embodiments, the CPS is calculated from a stained tumor tissue biopsy section (e.g., on a slide) or serial tumor tissue biopsy sections by immunohistochemistry (IHC) staining, in-situ hybridization (ISH; e.g., fluorescence-in-situ-hybridization, or FISH), histological stain, and a combination thereof. In certain embodiments, a tumor tissue biopsy section is analyzed by IHC to calculate the CPS. In certain embodiments, the percentage of viable PD-L1 positive and negative tumor cells and PD-L1 positive mononuclear inflammatory cells (MIC) is determined within the tumor nests and the adjacent supporting stroma. In such embodiments, cells are positive for PD-L1 staining if they display partial or complete membrane staining relative to all viable tumor cells present in the sample.

It is noted that patients with advanced-stage disease of certain tumor types (e.g., non-small cell lung carcinoma (NSCLC)) are often diagnosed on a small biopsy or cytology specimen obtained through a minimally invasive procedure. These small biopsy or cytology specimens are often the only samples available for testing. The methods described herein are especially attractive for cytology specimens such as these, i.e., those in which the context of the tissue architecture is lost, and it is very challenging to distinguish tumor from immune cells. Such samples include fine needle aspirates (where a thin needle is inserted into an area of abnormal-appearing tissue) or body fluid for sampling of cells. It can be the case that using CPS or a variation thereof is the only reliable method to analyze these specimens.

In other embodiments, the CPS is calculated from a tumor tissue sample that is not a fixed section on a slide. For example, in certain embodiments, the CPS is calculated using flow cytometric analysis of a cell suspension from the tumor tissue sample. In these embodiments, the tumor tissue cell suspension can be stained with a detectable PD-L1 binding agent (e.g., a fluorescently labeled antibody) and analyzed on a flow cytometer for counting the number of tumor cells and MIC cells expressing PD-L1. Tumor cells and MIC cells in the sample can be distinguished using any convenient flow cytometric parameter, e.g., forward scatter (FS), side scatter (SS), or by the expression of one or more additional markers using corresponding detectable binding agents for the one or more additional markers, e.g., markers specific or MIC or tumor cells. In other embodiments, the cells in the tumor tissue sample can be analyzed on a cell-by-cell basis for mRNA expression of PD-L1 and any other desired target, e.g., using single-cell nucleic acid sequencing methods for gene expression profiling (e.g., next generation sequencing methods).

As noted above, certain embodiments of the disclosed methods include staining the tumor tissue biopsy section for at least one (or multiple) additional target(s) or with a stain. Where multiple targets are assessed, e.g., a cell type-specific marker or a second tumor cell marker, the staining process may be done in a multiplex fashion, i.e., all desired markers are assessed on the same tissue sample simultaneously (e.g., using delectably distinguishable target-specific binding agents). In other embodiments, the multiple targets are detected on separate tissue samples, e.g., serial tissue sections derived from the same tumor biopsy from the subject. In embodiments where an additional stain is employed, the stain can be a histological stain, including but not limited to hematoxylin and eosin (H&E stain), which is the most commonly used light microscopy stain in histology and histopathology. Hematoxylin, a basic dye, stains nuclei blue due to an affinity to nucleic acids in the cell nucleus; eosin, an acidic dye, stains the cytoplasm pink.

Another commonly performed histochemical technique is the Peris Prussian blue reaction, used to demonstrate iron deposits in diseases like hemochromatosis. In some embodiments, the tissue sample is stained with a viability stain or dye to enhance the identification of viable cells. For example, a tissue sample analyzed by flow cytometry can be contacted with a viability dye prior to analysis, e.g., propidium iodide. Any convenient viability stain may be employed, with many examples known in the art.

Further, there are many other staining techniques known to those of skill in the art that can be used to selectively stain cells and cellular components that find use in the present disclosure, and as such no limitation in this regard is intended.

The staining of a target (e.g., PD-L1) in cells from a tumor tissue biopsy is generally done by contacting the cells with one or more detectable target-specific binding agents under suitable conditions to allow for binding of the target-specific binding agent to its desired target (while minimizing nontarget binding). As noted above, the term “target-specific binding agent” means any agent that specifically binds to a target or analyte of interest, e.g., a target of interest that is present in a tissue section as described herein (e.g., a polypeptide or polynucleotide). In some embodiments, the target-specific binding agent is an antibody (or target-binding fragments thereof), e.g., as used in IHC and flow cytometry.

Staining may be performed with primary and secondary antibodies or without using secondary antibodies (e.g., where the primary antibody is delectably labeled). Non limiting examples of anti-PD-L1 antibodies include, but are not limited to, clone 22C3 (Merck & Co.), clone 28-8 (Bristol-Myers Squibb), and clones SP263 or SP142 (Spring Biosciences). In certain other embodiments, the target specific binding agent is a nucleic acid or nucleic acid binding agent, e.g., as employed in in situ hybridization (ISH) reactions. For example, the target binding reagent can be a DNA, RNA, DNA/RNA hybrid molecule, peptide nucleic acid (PNA), and the like. No limitation in the metes and bounds of a target-specific binding agent that finds use in the subject disclosure is intended.

The target-specific binding agent (or any secondary reagent used to detect the target-specific binding agent) may be attached to any suitable detectable label (or chromogen) or enzyme capable of producing a detectable label. Thus, in certain embodiments, the first or second label is produced by an enzymatic reaction, e.g., by the activity of horseradish peroxidase, alkaline phosphatase, and the like. Any convenient enzymatic label/chromogen deposition system can be employed (e.g., as used in standard IHC methods), and as such, no limitation in this regard is intended. In some embodiments, the detectable label is a fluorescent tag.

In some embodiments, the staining reagents used may include a target-specific antibody (e.g., a PD-L1 specific antibody). Where an additional target is to be detected, the staining reagents used may include one or more additional antibodies that each bind to a different antigen. For example, a set of antibodies may include a first antibody that binds to a first antigen (e.g., PD-L1), a second antibody that binds to a second antigen, optionally a third antibody that binds to a third antigen and, optionally a fourth antibody that binds to a fourth antigen and/or further antibodies that bind to further antigens. In some embodiments, the antibody/antibodies used are primary antibodies that are detected by use of a secondary antibody (or other reagent). The staining steps thus may be done by incubating the cells of the tissue sample, e.g., a tissue section or cell suspension, with the primary antibody/antibodies and then, after the primary antibody has bound to the desired target in/on the cells, incubating the cells with the labeled secondary antibody/antibodies (e.g., as is done in standard IHC protocols). In some multiplex embodiments, each of the primary antibodies for each different target is from a different species (e.g., goat, rabbit, mouse, camel, chicken, donkey, etc.) and the corresponding secondary antibodies specific for each different primary antibody are distinguishably labeled from each other.

In some multiplex embodiments, the first and second (and optionally subsequent) targets being detected are different from each other, e.g., are different proteins or polynucleotides (e.g., different genes). However, in some multiplex embodiments, there may be some overlap. For example, in certain cases, a first target-specific binding agent may bind to the same target as a second target-specific binding agent but at a different epitope or site.

In certain embodiments, the sample being analyzed is a tissue section, e.g., a formalin fixed and paraffin embedded (FFPE) tissue section. In alternative embodiments, the tissue section has been fixed in a different way, including tissue sections that have been fixed in, e.g., acrolein, glyoxal, osmium tetroxide, carbodiimide, mercuric chloride, zinc salts, picric acid, potassium dichromate, ethanol, methanol, acetone, and/or acetic acid.

In embodiments in which a tissue section is analyzed by microscopy, the method further comprises comparing the relative location of the detected first (or any subsequent) label on the tissue section(s). This can be done, for example, by overlaying multiple images of the slide or series of slides that were collected during the analysis (e.g., for different labels). For example, one or more images collected for the labels of a first tissue section (or first label) can be overlaid onto one or more images collected for a second adjacent tissue section (or second, distinguishable label).

In certain embodiments, the images may be overlaid and analyzed to identify the boundaries of individual cells or regions in the tissue section, and/or subcellular features in individual cells, in the image. Computer-implemented methods for segmenting images of cells and tissues are known in the art and range from relatively simple thresholding techniques (see, e.g., Korde et al. Anal Quant Cytol Histol. 2009 31: 83-89 and Tuominen et al. Breast Cancer Res. 2010 12: R56), to more sophisticated methods, such as, for instance, adaptive attention windows defined by the maximum cell size (Ko et al. J Digit Imaging. 2009 22: 259-274) or gradient flow tracking (Li, et al. J Microsc. 2008 231: 47-58). Some suitable image segmentation methods may be reviewed in Ko et al. (J Digit Imaging. 2009 22: 259-74) and Ong et al. (Comput Biol Med. 1996 26:269-79). Next the data that corresponds to each of the individual parameters that have been defined by the segmenting are integrated to provide, for each cell, values that indicate which markers are associated with the cell. In certain cases, a cell may be identified as being malignant, non-malignant, infiltrating non-malignant, etc., as a result of this analysis. This data may allow one to potentially type the cells in the sample. As such, this method may comprise displaying an image of the sample, in which the cells are color-coded by their type.

In certain embodiments, the tissue section may be a section of a tissue biopsy obtained from a subject, e.g., a subject having a malignancy. Biopsies of interest include both tumor and non-neoplastic biopsies of skin (melanomas, carcinomas, etc.), soft tissue, bone, breast, colon, liver, kidney, adrenal, gastrointestinal, pancreatic, gall bladder, salivary gland, cervical, ovary, uterus, testis, prostate, lung, thymus, thyroid, parathyroid, pituitary (adenomas, etc.), brain, spinal cord, ocular, nerve, and skeletal muscle, etc. In certain embodiments, the subject from which the biopsy is obtained has a malignancy is selected from: gastric cancer, esophageal carcinomas, head and neck cancer (e.g., head and neck squamous cell carcinoma, or HNSCC), renal cell carcinoma, urothelial/bladder carcinoma, ovarian carcinoma, myeloma, melanoma, lung cancer, classical Hodgkin lymphoma, and breast cancer (e.g., triple negative breast cancer, hormone receptor positive (ER and/or PR) and Her2 positive breast cancer), small cell lung cancer, salivary gland carcinoma, vulvar carcinoma, thyroid carcinoma, anal canal carcinoma, biliary carcinoma, mesothelioma, cervical carcinoma, and neuroendocrine carcinoma.

In some embodiments, the method may involve obtaining one or more image as described above (e.g., an electronic form of which may have been forwarded from a remote location) and may be analyzed by a doctor or other medical professional to calculate the CPS. In other embodiments, the tissue sections are assessed in real time, i.e., not from a stored image of the slide (or other form of stored data). In some embodiments, a slide or image of the slide, as described above, is assessed and a CPS is calculated in an automated fashion in silico, e.g., without the slide(s) or image(s) of the slide being assessed by a human. In such embodiments, the slide(s)/image(s) are analyzed by a computer that has been programmed to analyze the staining pattern and identify the PD-L1 positive and negative tumor cells as well as the PD-L1 positive MICs. In other embodiments, the image of the slide or slides is annotated by a slide imaging device such that cells of each different cell type (e.g., PD-L1 positive tumor, PD-L1 negative tumor, and PD-L1 positive MIC) are readily identifiable by a user, e.g., by color coding of the cells or regions in the image.

In any embodiment, data can be forwarded to a “remote location,” where “remote location” means a location other than the location at which the image is examined. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but be separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or include email transmissions and information recorded on websites and the like. In certain embodiments, the image may be analyzed by a medical examiner or other qualified medical professional, and a report based on the results of the analysis of the image may be forwarded to the patient from which the sample was 10 obtained.

Unless defined otherwise herein, all technical and scientific terms used herein have the same meaning as commonly understood by one of ordinary skill in the art. Although any methods and materials similar or equivalent to those described herein can be used in the practice or testing disclosed herein, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively. The headings provided herein are not limitations of the various aspects or embodiments disclosed herein. Accordingly, the terms defined immediately below are more fully defined by reference to the specification as a whole.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989).

Unless otherwise indicated, “antibody fragment” or “antigen binding fragment” refers to a fragment of an antibody that retains the ability to bind specifically to the antigen, e.g., fragments that retain one or more CDR regions. An antibody that “specifically binds to” PD-1 or ILT4 is an antibody that exhibits preferential binding to PD-1 or ILT4 (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., PD-1 or ILT4). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The following references relate to “BLAST” algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

The terms “combination therapy” and “therapeutic combination” refer to treatments in which an anti-human ILT4 monoclonal antibody or antigen-binding fragment thereof, and an anti-human PD-1 monoclonal antibody or antigen-binding fragment thereof, are administered to a patient in a coordinated manner, over an overlapping period of time. The period of treatment with the anti-human PD-1 monoclonal antibody (or antigen-binding fragment thereof) (the “anti-PD-1 treatment”) is the period of time that a patient undergoes treatment with the anti-human PD-1 monoclonal antibody (or antigen-binding fragment thereof); that is, the period of time from the initial dosing with the anti-human PD-1 monoclonal antibody (or antigen-binding fragment thereof) through the final day of a treatment cycle. Similarly, the period of treatment with the anti-human ILT4 monoclonal antibody (or antigen-binding fragment thereof) (the “anti-ILT4 treatment”) is the period of time that a patient undergoes treatment with the anti-human ILT4 monoclonal antibody (or antigen-binding fragment thereof); that is, the period of time from the initial dosing with the anti-human ILT4 monoclonal antibody (or antigen-binding fragment thereof) through the final day of a treatment cycle. In the methods and therapeutic combinations described herein, the anti-PD-1 treatment overlaps by at least one day with the anti-ILT4 treatment. In certain embodiments, the anti-PD-1 treatment and the anti-ILT4 treatment are the same period of time. In some embodiments, the anti-PD-1 treatment begins prior to the anti-ILT4 treatment. In other embodiments, the anti-PD-1 treatment begins after the anti-ILT4 treatment. In certain embodiments, the anti-PD-1 treatment is terminated prior to termination of the anti-ILT4 treatment. In other embodiments, the anti-PD-1 treatment is terminated after termination of the anti-ILT4 treatment.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“ILT4 antagonist” or “anti-ILT4 agent” means any chemical compound or biological molecule that blocks binding of ILT4 to HLA-G, HLA-A, HLA-B, HLA-F, or angiopoietin-like protein (ANGPTL, such as ANGPTL1, ANGPTL4, or ANGPTL7). Alternative names or synonyms for ILT4 and its ligands include but are not limited to: ILT-4, leukocyte immunoglobulin-like receptor subfamily B member 2 (LILRB2), MIR10, MIR-10, LIR2, LIR-2, CD85D for ILT4; MHC-G or major histocompatibility complex, class I, G for HLA-G; major histocompatibility complex, class I, A for HLA-A; AS, B-4901, major histocompatibility complex, class I, B for HLA-B; CDA12, HLA-CDA12, or major histocompatibility complex, class I, F for HLA-F; angiopoietin-3, ANG3, ANGPT3, ARP1, UNQ162, angiopoietin like 1 for ANGPTLT; ARP4, HFARP, PGAR, UNQ171, angiopoietin like 4 for ANGPTL4; and CDT6, angiopoietin like 7 for ANGPTL7. In any of the treatment methods, medicaments and disclosed uses in which a human individual is being treated, the ILT4 antagonist blocks binding of human ILT4 to human HLA-G, HLA-A, HLA-B, HLA-F, ANGPTL1, ANGPTL4, or ANGPTL7. Human ILT4 precursor amino acid sequence can be found in NCBI Locus No.: AAB88119.1. Human HLA-G, HLA-A, HLA-B, and HLA-F precursor amino acid sequences can be found in NCBI Locus No.: P17693.1, P04439.2, P01889.3, P30511.3, respectively. Human ANGPTL1, ANGPTL4, and ANGPTL7 precursor amino acid sequences can be found in NCBI Locus No.: NP_001363692, Q9BY76.2, and 043827.1, respectively.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“PD-1 antagonist” or “anti-PD-1 agent” means any chemical compound or biological molecule that blocks binding of PD-L1 to PD-1 and preferably also blocks binding of PD-L2 to PD-1. Alternative names or synonyms for PD-1 and its ligands include: PDCD1, PD1, CD279 and SLEB2 for PD-1; PDCDILI, PDL1, B7H1, B7-4, CD274 and B7-H for PD-Li; and PDCD1L2, PDL2, B7-DC, Btdc and CD273 for PD-L2. In any of the treatment methods, medicaments and disclosed uses in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

“RECIST 1.1 Response Criteria” as used herein means the definitions set forth in Eisenhauer, E. A. et al., Eur. J Cancer 45:228-247 (2009) for target lesions or nontarget lesions, as appropriate based on the context in which response is being measured.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorphs (e.g., rabbits), rodents (e.g., rats or mice), Procyon lotor (e.g., raccoons). In particular embodiments, the subject is human.

The term “subject in need thereof” as used herein refers to a subject diagnosed with or suspected of having cancer or an infectious disease as defined herein.

“Sustained response” means a sustained therapeutic effect after cessation of treatment as described herein. In some embodiments, the sustained response has a duration that is at least the same as the treatment duration, or at least 1.5, 2.0, 2.5 or 3 times longer than the treatment duration.

“Treat” or “treating” cancer as used herein means to administer a monotherapy of an ILT4 antagonist or a combination therapy of an ILT4 antagonist and a PD-1 antagonist to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth. Such “treatment” may result in a slowing, interrupting, arresting, controlling, or stopping of the progression of cancer as described herein but does not necessarily indicate a total elimination of the cancer or the symptoms of the cancer. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination therapy of the disclosure is any of PR, CR, OR, PFS, DFS, and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or OR that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The monotherapy or combination therapy agents and compositions provided herein can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The terms “treatment regimen,” “dosing protocol,” and “dosing regimen” are used interchangeably to refer to the dose and timing of administration of each therapeutic agent in a combination therapy of the disclosure.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AML)), lymphoma (such as DLBCL), multiple myeloma (MM), etc.).

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “VH.” The variable region of the light chain may be referred to as “VL.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FRI, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

The term “variant” when used in relation to an antibody (e.g., an anti-PD-1 antibody or an anti-ILT4 antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-PD-1 antibody variant or an anti-ILT4 antibody variant) at least retains the antibody functional activity. In specific embodiments, an anti-PD-1 antibody variant binds to PD-1 and/or is antagonistic to PD-1 activity. In some embodiments, an anti-ILT4 antibody variant binds to ILT4 and/or is antagonistic to ILT4 activity.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

### ILT4 Antagonists (or Anti-ILT4 Agents)

Provided herein are ILT4 antagonists or anti-ILT4 agents that can be used in the various methods, kits, and uses disclosed herein, including any chemical compound or biological molecule that blocks binding of ILT4 to HLA-G, HLA-A, HLA-B, HLA-F, and/or ANGPTL (such as ANGPTL1, ANGPTL4, or ANGPTL7).

In one embodiment, an anti-ILT4 agent is an anti-ILT4 monoclonal antibody. Any monoclonal antibodies that bind to an ILT4 polypeptide, an ILT4 polypeptide fragment, an ILT4 peptide, or an ILT4 epitope and block the interaction between ILT4 and HLA-G, HLA-A, HLA-B, HLA-F, and/or ANGPTL (such as ANGPTL1, ANGPTL4, or ANGPTL7) can be used.

In certain embodiments of various methods, kits, or uses provided herein, the anti-human ILT4 monoclonal antibody or antigen binding fragment thereof comprises a VL CDR1, a VL CDR2, and a VL CDR3 comprising amino acid sequences as set forth in SEQ ID NOS: 1, 2, and 3, respectively, and a VH CDR1, a VH CDR2, and a VH CDR3 comprising amino acid sequences as set forth in SEQ ID NOS: 6, 7, and 8, respectively (Table 9).

In some embodiments of various methods, kits, or uses provided herein, the anti-human ILT4 monoclonal antibody or antigen binding fragment thereof comprises a VL region comprising an amino acid sequence as set forth in SEQ ID NO: 4, and a VH region comprising an amino acid sequence as set forth in SEQ ID NO: 9 (Table 9).

In other embodiments of various methods, kits, or uses provided herein, the anti-human ILT4 monoclonal antibody or antigen binding fragment thereof comprises a light chain comprising or consisting of an amino acid sequence as set forth in SEQ ID NO: 5 and a heavy chain comprising or consisting of an amino acid sequence as set forth in SEQ ID NO: 10 (Table 9).

In some embodiments, the anti-human ILT4 monoclonal antibody can be any antibody, antigen binding fragment thereof, or variant thereof disclosed in WO 2018/187518 and WO 2019/126514, the disclosures of which are incorporated by reference herein in their entireties.

In various embodiments, the anti-human ILT4 monoclonal antibody or antigen binding fragment thereof comprises a variant of the amino acid sequences of the anti-ILT4 antibodies disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human ILT4 monoclonal antibody or antigen binding fragment thereof has a VL domain with at least 95%, 90%, 85%, 80%, 75% or 50% sequence homology to one of the VL domains of the anti-ILT4 antibodies described herein, and exhibits specific binding to ILT4. In another embodiment, the anti-human ILT4 monoclonal antibody or antigen binding fragment thereof has a VH domain with at least 95%, 90%, 85%, 80%, 75% or 50% sequence homology to one of the VH domains of the anti-ILT4 antibodies described herein, and exhibits specific binding to ILT4. In yet another embodiment, the anti-human ILT4 monoclonal antibody or antigen binding fragment thereof has a VL domain with at least 95%, 90%, 85%, 80%, 75% or 50% sequence homology to one of the VL domains of the anti-ILT4 antibodies described herein and a VH domain with at least 95%, 90%, 85%, 80%, 75% or 50% sequence homology to one of the VH domains of the anti-ILT4 antibodies described herein, and exhibits specific binding to ILT4.

In one embodiment, the anti-human ILT4 monoclonal antibody or antigen binding fragment thereof has a VL domain having up to 1, 2, 3, 4, 5 or more amino acid substitutions, deletions and/or additions in one of the VL domains of the anti-ILT4 antibodies described herein, and exhibits specific binding to ILT4. In another embodiment, the anti-human ILT4 monoclonal antibody or antigen binding fragment thereof has a VH domain having up to 1, 2, 3, 4, 5 or more amino acid substitutions, deletions, and/or additions in one of the VH domains of the anti-ILT4 antibodies described herein, and exhibits specific binding to ILT4. In another embodiment, the anti-human ILT4 monoclonal antibody or antigen binding fragment thereof has a VL domain having up to 1, 2, 3, 4, 5 or more amino acid substitutions, deletions, and/or additions in one of the VL domains of the anti-ILT4 antibodies described herein and a VH domain having up to 1, 2, 3, 4, 5 or more amino acid substitutions, deletions, and/or additions in one of the VH domains of the anti-ILT4 antibodies described herein, and exhibits specific binding to ILT4. In yet another embodiment, the anti-human ILT4 monoclonal antibody or antigen binding fragment thereof has up to 1, 2, 3, 4, 5 or more amino acid substitutions, deletions, and/or additions outside the VL domains of the anti-human ILT4 antibodies described herein and up to 1, 2, 3, 4, 5 or more amino acid substitutions, deletions, and/or additions outside the VH domains of the anti-human ILT4 antibodies described herein, and exhibits specific binding to ILT4.

In various embodiments, the anti-human ILT4 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the VL and VH regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-ILT4 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, VH, VL, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments” in Theoretical and Computational Methods in Genome Research (S. Suhai, ed.), (1997) pp. 1-14, Plenum, New York.

In some embodiments, the anti-human ILT4 monoclonal antibody is a human antibody. In other embodiments, the anti-human ILT4 monoclonal antibody is a humanized antibody.

In some embodiments, the light chain of the anti-human ILT4 monoclonal antibody has a human kappa backbone. In other embodiments, the light chain of the anti-human ILT4 monoclonal antibody has a human lambda backbone.

In some embodiments, the heavy chain of the anti-human ILT4 monoclonal antibody has a human IgG1 backbone. In other embodiments, the heavy chain of the anti-human ILT4 monoclonal antibody has a human IgG2 backbone. In yet other embodiments, the heavy chain of the anti-human ILT4 monoclonal antibody has a human IgG3 backbone. In still other embodiments, the heavy chain of the anti-human ILT4 monoclonal antibody has a human IgG4 backbone.

In some embodiments, the heavy chain of the anti-human ILT4 monoclonal antibody has a human IgG1 variant backbone. In other embodiments, the heavy chain of the anti-human ILT4 monoclonal antibody has a human IgG2 variant backbone. In yet other embodiments, the heavy chain of the anti-human ILT4 monoclonal antibody has a human IgG3 variant backbone. In still other embodiments, the heavy chain of the anti-human ILT4 monoclonal antibody has a human IgG4 variant (e.g., IgG4 with S228P mutation) backbone.

In certain embodiments, the ILT4 antagonist is a molecule that binds to HLA-G, HLA-A, HLA-B, HLA-F, ANGPTL1, ANGPTL4, or ANGPTL7 and blocks the binding of ILT4 to HLA-G, HLA-A, HLA-B, HLA-F, ANGPTL1, ANGPTL4, or ANGPTL7. In one embodiment, the ILT4 antagonist is a molecule that binds to HLA-G and blocks the binding of ILT4 to HLA-G. In one embodiment, the ILT4 antagonist is a molecule that binds to HLA-A and blocks the binding of ILT4 to HLA-A. In one embodiment, the ILT4 antagonist is a molecule that binds to HLA-B and blocks the binding of ILT4 to HLA-B. In one embodiment, the ILT4 antagonist is a molecule that binds to HLA-F and blocks the binding of ILT4 to HLA-F. In one embodiment, the ILT4 antagonist is a molecule that binds to ANGPTL1 and blocks the binding of ILT4 to ANGPTL1. In one embodiment, the ILT4 antagonist is a molecule that binds to ANGPTL4 and blocks the binding of ILT4 to ANGPTL4. In one embodiment, the ILT4 antagonist is a molecule that binds to ANGPTL7 and blocks the binding of ILT4 to ANGPTL7. In some embodiments, the molecule that binds to HLA-G, HLA-A, HLA-B, HLA-F, ANGPTL1, ANGPTL4, or ANGPTL7 is a monoclonal antibody specifically binding to HLA-G, HLA-A, HLA-B, HLA-F, ANGPTL1, ANGPTL4, or ANGPTL7.

In one embodiment, the anti-ILT4 agent or ILT4 antagonist is Antibody A. In one embodiment, the anti-ILT4 agent or ILT4 antagonist comprises a VL sequence of SEQ ID No. 4 and a VH sequence of SEQ ID No. 9. In one embodiment, the anti-ILT4 agent or ILT4 antagonist comprises a VL-CDR1 of SEQ ID No. 1, a VL-CDR2 of SEQ ID No. 2, and a VL-CDR3 of SEQ ID No. 3; and a VH-CDR1 of SEQ ID No. 6, a VH-CDR2 of SEQ ID No. 7, and a VH-CDR3 of SEQ ID No. 8.

### PD-1 Antagonists (or Anti-PD-1 Agents)

As used herein, PD-1 antagonists or anti-PD-1 agents that can be used in the various methods, kits, and uses disclosed herein, include any chemical compound or biological molecule that blocks binding of PD-L1 to PD-1 and preferably also blocks binding of PD-L2 to PD-1.

In one embodiment, an anti-PD-1 agent is an anti-PD-1 monoclonal antibody. Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

Any monoclonal antibodies that bind to a PD-L1 polypeptide, a PD-L1 polypeptide fragment, a PD-L1 peptide, or a PD-L1 epitope and block the interaction between PD-L1 and PD-1 can also be used.

In certain embodiments, the anti-human PD-1 monoclonal antibody is selected from the group consisting of pembrolizumab, nivolumab, dostarlimab, cemiplimab, AMP-514 (MedImmune LLC, Gaithersburg, MD), PDR001 (U.S. Pat. No. 9,683,048), BGB-A317 (U.S. Pat. No. 8,735,553), and MGA012 (MacroGenics, Rockville, MD). In one embodiment, the anti-human PD-1 monoclonal antibody is pembrolizumab. In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is dostarlimab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In some embodiments, the anti-human PD-1 monoclonal antibody can be any antibody, antigen binding fragment thereof, or variant thereof disclosed in U.S. Pat. Nos. 7,488,802, 7,521,051, 8,008,449, 8,354,509, 8,168,757, WO2004/004771, WO2004/072286, WO2004/056875, US2011/0271358, and WO 2008/156712, the disclosures of which are incorporated by reference herein in their entireties.

Examples of monoclonal antibodies that bind to human PD-L1 that can be used in various methods, kits, and uses described herein are disclosed in WO2013/019906, WO2010/077634, and U.S. Pat. No. 8,383,796, the disclosures of which are incorporated by reference herein in their entireties. Specific anti-human PD-L1 monoclonal antibodies useful as the PD-1 antagonist in the various methods, kits, and uses described include atezolizumab, durvalumab, avelumab, BMS-936559, and an antibody comprising the heavy chain and light chain variable regions of SEQ ID NO:20 and SEQ ID NO:21, respectively, of WO2013/019906.

Other PD-1 antagonists useful in various methods, kits, and uses described herein include an immunoadhesion molecule that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, the disclosures of which are incorporated by reference herein in their entireties. Specific fusion proteins useful as the PD-1 antagonist in various methods, kits, and uses described herein include AMP-224 (also known as B7-DCIg), which is a PD-L2-Fc fusion protein and binds to human PD-1.

In various embodiments, the anti-human PD-1 or anti-human PD-L1 monoclonal antibody or antigen binding fragment thereof comprises a variant of the amino acid sequences of the anti-human PD-1 or anti-human PD-L1 antibodies described herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human PD-1 or anti-human PD-L1 monoclonal antibody or antigen binding fragment thereof has a VL domain with at least 95%, 90%, 85%, 80%, 75% or 50% sequence homology to one of the VL domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a VH domain with at least 95%, 90%, 85%, 80%, 75% or 50% sequence homology to one of the VH domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a VL domain with at least 95%, 90%, 85%, 80%, 75% or 50% sequence homology to one of the VL domains of the anti-human PD-1 or anti-human PD-L1 antibodies described herein and a VH domain with at least 95%, 90%, 85%, 80%, 75% or 50% sequence homology to one of the VH domains of the anti-human PD-1 or anti-human PD-L1 antibodies described herein, and exhibits specific binding to PD-1 or PD-L1.

In one embodiment, the anti-human PD-1 or anti-human PD-L1 monoclonal antibody or antigen binding fragment thereof has a VL domain having up to 1, 2, 3, 4, 5 or more amino acid substitutions, deletions and/or additions in one of the VL domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a VH domain having up to 1, 2, 3, 4, 5 or more amino acid substitutions, deletions, and/or additions in one of the VH domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a VL domain having up to 1, 2, 3, 4, 5 or more amino acid substitutions, deletions, and/or additions in one of the VL domains of the anti-human PD-1 or anti-human PD-L1 antibodies described herein and a VH domain having up to 1, 2, 3, 4, 5 or more amino acid substitutions, deletions, and/or additions in one of the VH domains of the anti-human PD-1 or anti-human PD-L1 antibodies described herein, and exhibits specific binding to PD-1 or PD-L1. In a particular embodiment, the anti-human PD-1 or anti-human PD-L1 monoclonal antibody or antigen binding fragment thereof has a VL domain having up to 1, 2, 3, 4, 5 or more amino acid substitutions, deletions, and/or additions in the framework region of one of the VL domains of the anti-human PD-1 or anti-human PD-L1 antibodies described herein and a VH domain having up to 1, 2, 3, 4, 5 or more amino acid substitutions, deletions, and/or additions in the framework region of one of the VH domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has up to 1, 2, 3, 4, 5 or more amino acid substitutions, deletions, and/or additions outside the VL domains of the anti-human PD-1 or anti-human PD-L1 antibodies described herein and up to 1, 2, 3, 4, 5 or more amino acid substitutions, deletions, and/or additions outside the VH domains of the anti-human PD-1 or anti-human PD-L1 antibodies described herein, and exhibits specific binding to PD-1 or PD-L1.

In various embodiments, the anti-human PD-1 or anti-human PD-L1 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the VL and VH regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-human PD-1 or anti-human PD-L1 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, VH, VL, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human PD-1 or anti-human PD-L1 monoclonal antibody is a human antibody. In other embodiments, the anti-human PD-1 or anti-human PD-L1 monoclonal antibody is a humanized antibody.

In some embodiments, the light chain of the anti-human PD-1 or anti-human PD-L1 monoclonal antibody has a human kappa backbone. In other embodiments, the light chain of the anti-human PD-1 or anti-human PD-L1 monoclonal antibody has a human lambda backbone.

In some embodiments, the heavy chain of the anti-human PD-1 or anti-human PD-L1 monoclonal antibody has a human IgG1 backbone. In other embodiments, the heavy chain of the anti-human PD-1 or anti-human PD-L1 monoclonal antibody has a human IgG2 backbone. In yet other embodiments, the heavy chain of the anti-human PD-1 or anti-human PD-L1 monoclonal antibody has a human IgG3 backbone. In still other embodiments, the heavy chain of the anti-human PD-1 or anti-human PD-L1 monoclonal antibody has a human IgG4 backbone.

In some embodiments, the heavy chain of the anti-human PD-1 or anti-human PD-L1 monoclonal antibody has a human IgG1 variant backbone. In other embodiments, the heavy chain of the anti-human PD-1 or anti-human PD-L1 monoclonal antibody has a human IgG2 variant backbone. In yet other embodiments, the heavy chain of the anti-human PD-1 or anti-human PD-L1 monoclonal antibody has a human IgG3 variant backbone. In still other embodiments, the heavy chain of the anti-human PD-1 or anti-human PD-L1 monoclonal antibody has a human IgG4 variant (e.g., IgG4 with S228P mutation) backbone.

In one embodiment, the anti-PD-1 agent or PD-1 antagonist is pembrolizumab.

The following examples are intended to promote a further understanding of the present disclosure.

### Example 1

This example is to provide guidelines for evaluating PD-L1 expression on formalin-fixed, 20 paraffin-embedded (FFPE) tumor tissue section with Dako's PD-L1 IHC 22C3 pharmDx kit (SK006). This immunohistochemical (IHC) assay is performed using the Dako Auto-stainer Link 48 automated staining system.

The embodiments below are described with respect to the use of the PD-L1 IHC 22C3 pharmDx kit, which is a qualitative immunohistochemical assay using Monoclonal Mouse Anti-PD-L1, Clone 22C3. This kit is intended for use in the detection of PD-L1 protein in formalin-fixed, paraffin-30 embedded (FFPE) tumor tissue using EnVision FLEX visualization system on Autostainer Link 48. Here, PD-L1 protein expression is used to determine a Combined Positive Score (CPS; as describe above). In these embodiments, the specimen is considered PD-L1 positive if the CPS>5, where PD-L1 positivity is defined as a viable cell exhibiting membrane staining with the 22C3 antibody at any intensity (as compared to positive and negative controls, as described below).

The following acronyms have the definitions listed in Table 3.

TABLE 3
Definitions of Acronyms

| Acronym | Definition for Acronym |
|---|---|
| CPS | Combined Positive Score |
| H&E | Hemotaxylin and eosin |
| MCF-7 | PD-L1-negative control cell line |
| MIC | Mononuclear inflammatory cells |
| NCI-H226 | PD-L1-positive control cell line |
| NCR | Negative Control Reagent |
| PD-L1 | Programmed Death Ligand 1 |

Clinical Interpretation Guidelines for PD-L1 IHC 22C3 pharmDx in Tumor Tissue Specimen Criteria

A hemotaxylin and eosin (H&E) stained section is recommended for the evaluation of an acceptable tumor tissue sample. PD-L1 IHC 22C3 pharmDx and the H&E staining is performed on serial sections from the same paraffin block of the specimen to confirm:
1. The histological diagnosis of the cancer/malignancy.
2. The specimen contains a minimum of 100 viable tumor cells to determine the percentage of positive cells. For specimens with less than 100 viable tumor cells, tissue from a deeper level of the block, or potentially another block, could present sufficient number of viable tumor cells for PD-L1 IHC 22C3 pharmDx testing.
3. The specimen has been properly fixed and prepared for IHC analysis. Well-preserved and well-stained areas of the specimen are used to make a determination of the percentage of cells present (e.g., PD-L1 positive tumor cells).

### Evaluating Controls

Deviations in the recommended procedures for tissue fixation, processing and embedding in the user's laboratory may produce significant variability in results. Therefore, the following controls in Table 4 can be included in each staining run.

TABLE 4
Quality Control Summary

| Control Type | Reagents Used in Testing | Purpose of Testing |
|---|---|---|
| Control Slide supplied by Dako | Primary Antibody, | Controls staining procedure |
| | *Negative Control Reagent | only |
| | & Detection System | |
| Positive Control: Tissue or cells | Primary Antibody, Negative | Controls all steps of the |
| containing target antigen to be | Control Reagent & | analysis. Validates reagents |
| detected. The ideal control is | Detection System | and procedures used for |
| weakly positive staining tissue, | | PD-L1 staining. |
| which may be more sensitive in | | |
| detecting reagent degradation. | | |
| Negative Control: Tissues or | Primary Antibody, Negative | Detection of unintended |
| cells expected to be negative | Control Reagent & | antibody cross-reactivity to |
| (could be located in patient | Detection System | cells/cellular components. |
| tissue or positive control tissue). | | |
| Patient tissue slide | Negative Control Reagent & | Detection of non-specific |
| | same Detection System as | background staining. |
| | used with the Primary | |
| | Antibody | |

*From the same species as the primary antibody, but not directed against the same target antigen. To detect non-specific antibody binding, e.g., binding of Fc portion of antibody by the tissue.
All control tissues must meet acceptance criteria to proceed with evaluation of PD-L1 staining on gastric carcinoma tissues.

PD-L1 IHC 22C3 pharmDx Control Cell Line Slide

Examine the PD-L1 IHC 22C3 pharmDx control cell line slide to determine that reagents are functioning properly. Each slide contains sections of cell pellets with positive and negative PD-L1 expression. Assess the percentage of positive cells and the staining intensity. If any staining of the control cell line slide is not satisfactory, all results with the specimens should be considered invalid. Control cell line slide is not used as an aid in interpretation of patient results.

### Evaluate the Overall Staining Intensity Using the Following Guide:

0 Negative
1+ Weak Intensity
2+ Moderate Intensity
3+ Strong Intensity

### User-Provided Control Tissue Slides

A user-provided positive control tissue slide is examined after the control cell line slides described above. This slide verifies that the fixation method and epitope retrieval process are effective. The ideal positive control tissue provides a complete dynamic representation of weak to moderate cell membrane staining of tumor cells and/or cell membrane/cytoplasmic staining of tumor-associated mononuclear inflammatory cells (MICs). Use intact cells for interpretation of staining results because necrotic or degenerated cells often stain non-specifically. Non-specific staining should be <1+.

A user-provided negative control tissue slide is also examined to verify the specificity of the labeling of the target antigen by the primary antibody. The ideal negative control tissue demonstrates no staining on tumor cells and MICs. The absence of specific staining in the negative control tissue slide confirms the lack of kit cross-reactivity to cells/cellular components. The variety of different cell types present in most tissue sections offers internal negative control sites; this should be verified by the user.

If staining of positive and/or negative control tissues are not satisfactory, the results with the patient specimen should be considered invalid.

In addition, patient specimens are stained with a Negative Control Reagent (NCR) from the PD-L1 IHC 22C3 pharmDx kit. Specimens stained with the NCR should have 0 specific staining and <1+ non-specific staining. Staining occurring in the cytoplasm of tumor cells of the specimen treated with the NCR should be considered non-specific staining.

### Example of Scoring Guidelines

For evaluation of tissue section staining and scoring (e.g., using IHC), an objective of 10-20× magnification is used. Partial or complete cell membrane staining of tumor cells that is perceived distinct from cytoplasmic staining is considered positive, as is cell membrane/cytoplasmic staining of MICs within the tumor nests and the adjacent supporting stroma. Adjacent MICs are defined as being within the same 20× field as the tumor. However, MICs that are NOT directly associated with the response against the tumor should be excluded. For example, in some cases, MICs are in the 20× field of view but can clearly be directly linked to non-tumor related causes. Such MICs should be excluded from scoring.

Refer to Table 5 below for details in evaluating PD-L1 positivity.

TABLE 5
Details in Evaluating PD-L1 Positivity

| Element | Included in Scoring | Excluded from Scoring |
|---|---|---|
| Tumor cells | Convincing partial or complete | Tumor cell cytoplasmic staining |
| | membrane staining (at any | |
| | intensity) of viable carcinoma | |
| | tumor cells | |
| Immune cells | Membrane and/or cytoplasmic* | Immune cells not infiltrating nor |
| | staining (at any intensity) of | adjacent to tumor |
| | mononuclear inflammatory cells | Normal cells adjacent to tumor cells |
| | (MIC) within tumor nests and | Stromal cells (fibroblasts) Necrotic |
| | adjacent supporting stroma**, | cells and/or cellular debris that may |
| | such as: Lymphocytes | stain PD-L1 positive |
| | (lymphocyte aggregates) | Plasma cells |
| | Monocytes Macrophages | Neutrophils |
| Morphology | N/A | Tissue damage/immune cells not |
| Patterns | | responsive to tumor (e.g., chronic |
| | | inflammation) |

*Lymphocytes often present indistinctive staining of membrane and cytoplasm due to a high nuclear to cytoplasmic ratio. Therefore, membrane and/or cytoplasmic staining in lymphocytes shall be included in the score.
**Adjacent MICs are defined as being within the same 20x field as the tumor. However, MICs that are NOT directly associated with the response against the tumor should be excluded.

### Example 2

Example 2 provides simple calculations of CPS values to better illustrate the instant disclosure.

Calculate CPS for a Specimen Containing 100 Total Tumor Cells, 80 Positive Tumor Cells, and 50 Positive Adjacent MIC Cells

$$CPS = \frac{80 positive tumor cells + 50 positive MIC cells}{100 total tumor cells} \times 100.$$

In this case the CPS=100 (although the score is greater than 100, the maximum CPS is defined as 100). The specimen is thus scored as PD-L1 Positive, and as such the subject is considered as eligible for anti-ILT4 and anti-PD-1 combination therapy.

Calculate CPS for a Specimen Containing 1000 Total Tumor Cells, 0 Positive Tumor Cells, 50 Positive Adjacent MIC Cells

$$CPS = \frac{0 positive tumor cells + 50 positive MIC cells}{1000 total tumor cells} \times 100.$$

In this case the CPS=5. The specimen is thus scored as PD-L1 Positive (the threshold is 5 in this example), and as such the subject is considered as eligible for anti-ILT4 and anti-PD-1 combination therapy.

Calculate CPS for a Specimen Containing 1000 Total Tumor Cells, 5 Positive Tumor Cells, and 0 Positive Adjacent MIC Cells

$$CPS = \frac{5 positive tumor cells + 0 positive MIC cells}{1000 total tumor cells} \times 100.$$

In this case the CPS=0.5. The specimen is thus scored as PD-L1 Negative, and as such the subject is not considered as eligible for anti-ILT4 and anti-PD-1 combination therapy.

### Example 3

Study Design—In a multicenter, open-label, first-in-human, phase I trial, patients were assigned to receive Antibody A as monotherapy (parts A, B) or in combination with pembrolizumab (part C). Study design details can be found at ClinicalTrials.gov, NCT03564691.

Patients whose disease progressed on monotherapy were eligible to cross over to combination therapy following clinical or radiographical evaluation. This study was conducted in accordance with Good Clinical Practice Guidelines and the Declaration of Helsinki; the protocol (4830-001-03) was approved by the institutional review boards or ethics committees of all participating sites. All patients provided written informed consent to participate before enrollment.

Patient Population—Patients (aged≥18 years) had histologically or cytologically confirmed metastatic solid tumors for which no available therapy could convey clinical benefit (previous anti-PD-1/PD-L1 therapy was permitted), measurable disease per RECIST (response evaluation criteria in solid tumors) v1.1, Eastern Cooperative Oncology Group performance status of 0 or 1, evaluable baseline tumor sample (archived or newly collected), and adequate organ function. Key exclusion criteria included chemotherapy, radiation therapy, or biological anticancer therapy≥4 weeks before the first dose, discontinuation of any previous immunotherapy regimen because of an immune-related AE, previous treatment with another agent targeting ILT4 or HLA-G, chronic systemic steroid therapy (the exception was oral physiological replacements), known untreated central nervous system metastasis or carcinomatous meningitis, and active autoimmune disease.

From Jul. 11, 2018 to Jul. 10, 2020, 99 patients were screened and 84 were treated in the dose-escalation phase of this study (Antibody A monotherapy, n=50; Antibody A plus pembrolizumab, n=34). Eighteen patients crossed over from the monotherapy group to receive combination therapy upon radiological confirmation of progressive disease (PD). The median age was 63 years (range, 31-83); most patients (56%) had previously received≥3 lines of therapy, and 23 patients (27%) had been previously treated with immunotherapy, including anti-PD-1/PD-L1 therapy.

In the monotherapy group, patients were treated with Antibody A at the following doses: 3 mg (n=2), 10 mg (n=2), 30 mg (n=1), 100 mg (n=7), 300 mg (n=8), 800 mg (n=15), and 1600 mg (n=15).

In the combination group, all patients received pembrolizumab 200 mg plus Antibody A at the following doses: 100 mg (n=5), 300 mg (n=6), 800 mg (n=8), and 1600 mg (n=15).

Median time from cycle 1, day 1 of treatment to data cutoff was 17 months (range, 11-24) in the monotherapy group and 13 months (range, 11-18) in the combination group.

At the data cutoff, median treatment duration was 1 month (range, 0-16) in the monotherapy group and 4 months (range, 0-15) in the combination group; 46 patients (PD, n=43; adverse event [AE], n=2; patient withdrawal, n=1) and 25 patients (PD, n=24; AE, n=1) discontinued study treatment, respectively.

Treatment—Antibody A was administered intravenously at a starting dose of 3 mg and subsequent dose escalation to a maximum proposed dose of 1600 mg every 3 weeks (Q3W). Given the potential for Antibody A to activate the immune system and the limitations of standard toxicology studies to model these effects in the preclinical setting, a conservative starting dose was chosen to ensure safety. The 3 mg starting dose of Antibody A was determined based on integration of the data obtained from in vitro studies evaluating Antibody A binding to granulocytes and monocytes obtained from healthy volunteers and patients with cancer and on nonclinical pharmacokinetics and safety and toxicology studies in nonhuman primates. Emphasis was placed on the in vitro receptor binding data because of the lack of target homology, orthologous protein expression, and cross-reactivity of Antibody A for ILT4 in rodents and nonhuman primates. The maximum proposed dose of 1600 mg was projected to provide pharmacokinetic exposures in the micromolar range. The highest dose of Antibody A was expected to provide>90% target receptor occupancy (RO) based on receptor-binding affinity and was to be confirmed by measuring blood RO.

Part A of the dose-escalation phase (Antibody A monotherapy) followed an accelerated titration design (ATD) with 1 to 3 patients treated per cohort with an about 3-fold increase between Antibody A dose levels. The starting dose for part B (Antibody A monotherapy) was based on safety criteria (DLT or grade≥2 toxicity) or ≥75% ILT4 RO in peripheral blood mononuclear cells demonstrated at any dose level in part A.

Part B of the dose-escalation phase continued using a modified toxicity probability interval (mTPI) design to identify the maximum tolerated dose MTD or the maximum administered dose (MAD) of Antibody A monotherapy.

Enrollment in part C (combination therapy with Antibody A plus pembrolizumab 200 mg Q3W) was initiated after the first 2 doses in part B were completed. Part C of the dose-escalation phase used the mTPI method to determine the MTD or MAD of Antibody A in combination with pembrolizumab. Treatment continued until PD, unacceptable AEs, intercurrent illness, investigator/patient decision to withdraw, or 2 years of treatment.

Study Outcomes—The primary objective of the dose-escalation phase was to characterize the safety and tolerability of Antibody A as monotherapy and in combination with pembrolizumab. The secondary objective was to evaluate pharmacokinetic parameters of Antibody A when administered alone or with pembrolizumab. Tertiary objectives included evaluation or identification of the following: circulating Antibody A and pembrolizumab antibodies; pharmacokinetics of pembrolizumab administered in combination with Antibody A; ORR as determined by RECIST v1.1 and immune-related RECIST per investigator (24); molecular (genomic, metabolic, proteomic, and/or transcriptomic) biomarkers potentially indicative of clinical response or resistance, safety, pharmacodynamic activity, or mechanism of action of Antibody A as monotherapy and in combination with pembrolizumab. Disease control rate (DCR) includes CR, PR, or SD with PFS duration of ≥6 months.

Safety Assessments and Imaging—The DLT evaluation period for the monotherapy and combination groups encompassed events that occurred within the first 3 weeks of cycle 1, day 1. Dose finding for parts B and C followed the mTPI design, with a target DLT rate of 30%. Safety was assessed by reviews of AEs and serious AEs during the study and for 30 days after the last dose if the patient transitioned to a new anticancer therapy or 90 days after the last dose if the patient remained on study.

For patients initially assigned to monotherapy, AEs were recorded in the monotherapy group if they occurred while the patient was receiving monotherapy and in the combination therapy group if they occurred after the patient crossed over to combination therapy. AE severity was graded according to the Common Terminology Criteria for Adverse Events, version 4.0.

Tumor imaging using computed tomography or magnetic resonance imaging was performed every 9 weeks (Q9W) until confirmed PD, start of new anticancer therapy, withdrawal of consent, death, or end of study.

No dose-limiting toxicities (DLTs) were observed, and a maximum-tolerated dose (MTD) was not reached in either the monotherapy or the combination therapy group.

In the monotherapy group, 50 patients (100%) experienced an adverse event (AE) (Table 1); the most common (≥20%) AEs were fatigue (40%), nausea (28%), decreased appetite (22%), and diarrhea (20%) (Table 2). Treatment-related AEs (TRAEs) of any grade occurred in 24 patients (48%) (Table 1); 3 patients (6%) experienced a total of 7 grade 3 or grade 4 events (fatigue, n=1; increased aspartate aminotransferase [AST], n=2; increased alanine aminotransferase [ALT], n=1; increased blood alkaline phosphatase, n=1; increased blood pressure, n=1; increased gamma-glutamyl transferase, n=1) (Table 2). One patient (2%) discontinued treatment because of a TRAE (increased AST, grade 2).

Of the 52 patients treated with combination therapy (n=34 in part C; n=18 who crossed over from parts A and B), 48 (92%) experienced AEs (Table 1); the most common (≥20%) AEs were decreased appetite (27%), fatigue (27%), and vomiting (21%) (Table 6). TRAEs of any grade occurred in 28 patients (54%) (Table 6); 4 patients (8%) experienced a grade 3 or grade 4 event (fatigue, n=1; pneumonitis, n=1; hyperglycemia, n=1; hypotension, n=1) (Table 7). One patient discontinued treatment because of a TRAE (pneumonitis). Four patients (8%) in the combination group experienced a serious TRAE; none led to discontinuation. No treatment-related deaths (grade 5 event) occurred in either treatment group.

TABLE 6
Adverse Event Summary

| | Antibody A | | Antibody A + | |
|---|---|---|---|---|
| | monotherapy^{a} | | pembrolizumab^{a} | |
| AE, n (%) | n = 50 (%) | | n = 52 (%) | |
| Any-grade AE | 50 | (100) | 48 | (92) |
| Grade 3-5 | 23 | (46) | 23 | (44) |
| Led to discontinuation | 2 | (4) | 1 | (2) |
| Serious | 14 | (28) | 18 | (35) |
| Serious and led to discontinuation | 0 | | 0 | |
| Led to death^{b} | 2 | (4) | 0 | |
| Any-grade TRAE | 24 | (48) | 28 | (54) |
| Grade 3-5 | 3 | (6) | 4 | (8) |
| Led to discontinuation | 1 | (2) | 1 | (2) |

| | | | |
|---|---|---|---|
| Serious | 0 | 4 | (8) |

| | | |
|---|---|---|
| Serious and led to discontinuation | 0 | 0 |
| Led to death | 0 | 0 |

Abbreviations: AE, adverse event; TRAE, treatment-related adverse event.
^{a}AEs occurring before crossover are counted in the monotherapy column; AEs occurring after crossover are counted in the combination therapy column.
^{b}AEs leading to death were death (n = 1) and tumor hemorrhage (n = 1).

TABLE 7
Most Common Adverse Events in Either Group

| | Antibody A | | Antibody A + | |
|---|---|---|---|---|
| | monotherapy^{a} | | pembrolizumab^{a} | |
| | n = 50 (%) | | n = 52 (%) | |
| AE, n (%) | Any | Grade ≥3 | Any | Grade ≥3 |

Most common AEs (≥10% incidence)

| Fatigue | 20 | (40) | 4 | (8) | 12 | (23) | 11 | (2) |
|---|---|---|---|---|---|---|---|---|
| Nausea | 12 | (24) | 0 | | 8 | (15) | 0 | |
| Decreased appetite | 11 | (22) | 1 | (2) | 14 | (27) | 0 | |
| Diarrhea | 10 | (20) | 0 | | 8 | (15) | 0 | |
| Abdominal pain | 8 | (16) | 2 | (4) | 8 | (15) | 2 | (4) |
| Dyspnea | 8 | (16) | 3 | (6) | 8 | (15) | 0 | |
| Arthralgia | 7 | (14) | 0 | | 7 | (13) | 0 | |
| Back pain | 7 | (14) | 0 | | 7 | (13) | 1 | (2) |
| Constipation | 7 | (14) | 0 | | 7 | (13) | 0 | |
| Cough | 7 | (14) | 0 | | 7 | (13) | 0 | |
| Pneumonia | 7 | (14) | 4 | (8) | 1 | (2) | 1 | (2) |
| Vomiting | 7 | (14) | 0 | | 11 | (21) | 0 | |
| Pyrexia | 6 | (12) | 0 | | 3 | (6) | 0 | |
| Upper abdominal pain | 5 | (10) | 0 | | 1 | (2) | 0 | |
| Anemia | 5 | (10) | 1 | (2) | 8 | (15) | 4 | (8) |

| | | | | | | |
|---|---|---|---|---|---|---|
| Dizziness | 5 | (10) | 0 | 4 | (8) | 0 |
| Headache | 5 | (10) | 0 | 6 | (12) | 0 |
| Peripheral edema | 1 | (2) | 0 | 9 | (17) | 0 |
| Hypothyroidism | 2 | (4) | 0 | 7 | (13) | 0 |
| Increased blood creatinine | 2 | (4) | | 6 | (12) | 0 |

Most common TRAEs (≥5% incidence)

| | | | | | | | | |
|---|---|---|---|---|---|---|---|---|
| Fatigue | 6 | (12) | 1 | (2) | 7 | (13) | 1 | (2) |

| Diarrhea | 5 | (10) | 0 | 2 | (4) | 0 | |
|---|---|---|---|---|---|---|---|
| Arthralgia | 4 | (8) | 0 | 3 | (6) | 1 | (2) |

| | | | | | | |
|---|---|---|---|---|---|---|
| Increased aspartate aminotransferase | 3 | (6) | 2 | (4) | 0 | 0 |

| | | | | | | |
|---|---|---|---|---|---|---|
| Decreased appetite | 2 | (4) | 0 | 1 | (2) | 0 |
| Nausea | 3 | (6) | 0 | 3 | (6) | 0 |
| Pruritus | 3 | (6) | 0 | 2 | (4) | 0 |
| Vomiting | 3 | (6) | 0 | 1 | (2) | 0 |
| Hypothyroidism | 2 | (4) | 0 | 5 | (10) | 0 |
| Maculopapular rash | 2 | (4) | 0 | 4 | (8) | 0 |

Abbreviations: AE, adverse event; TRAE, treatment-related adverse event.
^{a}AEs occurring before crossover are counted in the monotherapy column; AEs occurring after crossover are counted in the combination therapy column.

Pharmacokinetics, Anti-Drug Antibodies, and Target Engagement—Serum concentrations of Antibody A and pembrolizumab were used to derive pharmacokinetic parameters of Antibody A alone and with pembrolizumab. Blood samples were collected to evaluate levels of circulating ADAs and to perform pharmacodynamic RO analysis. RO of ILT4 was measured on circulating myeloid cells before and after the administration of Antibody A.

Briefly, whole blood and fresh tumor biopsy samples for ILT4 RO were assessed using fit-for-purpose validated flow cytometry assays to support exploratory biomarker endpoints. A dual detection method was used to measure unoccupied and total ILT4 on the cell surfaces of representative myeloid cell types before or after Antibody A administration or at both time points. In blood, CD45+/Lin1⁻/CD11b⁺/CD33⁺/HLADR⁺-expressing monocytes were assessed, whereas in fresh tumor samples, only viable CD45⁺/Lin1⁻/CD11b⁺/CD33⁺-expressing monocytes were assessed because of the variable sample quality.

Serum levels of Antibody A increased with increasing doses (3-1600 mg). Increases in Antibody A doses (3-1600 mg) resulted in dose-dependent increases in blood ILT4 receptor occupancy (RO). The initial Antibody A monotherapy dose escalation followed an accelerated titration design (ATD) to minimize the number of patients treated at potentially sub-therapeutic doses of Antibody A. The 100 mg starting dose for part B, which used the modified toxicity probability interval (mTPI) design with ≥3 patients per dose level, was considered appropriate to ensure some therapeutic benefit based on an average blood percentage RO of ≥60% at Antibody A C_{trough}.

In part B, an average blood RO of >80% was achieved at dose levels above 300 mg throughout the dosing interval. In patients with available Antibody A serum pharmacokinetic and blood RO data (n=21), the average blood percentage RO was 96.4%±3.62 at the end of cycle 1, i.e., cycle 2 pre-dose (day 21 pharmacokinetics showed C_{trough}=74.9±17.9 μg/mL) at the 800 mg dose level. Attempts to quantify tumor ILT4 RO from fresh tumor biopsy samples were not successful. Antibody A immunogenicity assessment preliminary results suggest a low incidence of anti-drug antibodies (ADAs). For Antibody A, a preliminary recommended phase 2 dose of 800 mg every 3 weeks (Q3W) was selected based on the entirety of the data, which included achieving>95% average blood RO in parts B and C of dose escalation.

Eleven of 84 patients with heavily pretreated advanced solid tumors in the dose-escalation phase of the trial achieved an objective response. One of 50 patients in the monotherapy of Antibody A achieved a confirmed partial response (PR) for an objective response rate (ORR) of 2% (Table 8); this patient had high-grade serous ovarian cancer and disease progression on 7 previous chemotherapy treatments and received Antibody A at the 800 mg dose level. Another patient with ovarian cancer in the monotherapy group completed 35 cycles of treatment and had a best overall response of stable disease (SD).

TABLE 8
Summary of Confirmed Tumor Response per
RECIST v1.1 by Investigator Assessment

| | | | | | Crossover to | |
|---|---|---|---|---|---|---|
| | Antibody A | | Antibody A + | | Antibody A + | |
| | monotherapy | | pembrolizumab | | pembrolizumab | |
| Confirmed response | n = 50 (%) | | n = 34 (%) | | n = 18 (%) | |
| ORR | 1 | (2)^{a} | 8 | (24)^{b} | 1 | (6)^{c} |
| CR | 0 | | 1 | (3) | 0 | |
| PR | 1 | (2) | 7 | (21) | 1 | (6) |
| SD | 11 | (22) | 9 | (26) | 1 | (6) |
| SD with PFS ≥6 months | 5 | (1) | 6 | (18) | 0 | |
| PD | 34 | (68) | 16 | (47) | 3 | (17) |
| No RECIST assessment^{d} | 4 | (8) | 1 | (3) | 13 | (72)^{e} |

Abbreviations: CR, complete response; ORR, objective response rate; PFS, progression-free survival; PR, partial response; RECIST v1.1, Response Evaluation Criteria in Solid Tumors version 1.1; SD, stable disease.
^{a}Patient had high-grade serous ovarian cancer.
^{b}Patients had head and neck squamous cell carcinoma (n = 2), gastric cancer (n = 2), Merkel cell carcinoma (n = 1), dendritic sarcoma (n = 1), non-small cell lung cancer (n = 1), and papillary thyroid (n = 1).
^{c}Patient had microsatellite-high colorectal cancer.
^{d}Includes patients who had the opportunity to have a postbaseline assessment based on the date of their first dose but did not have any postbaseline assessment by the data cut-off date.
^{e}11 patients have been evaluated by immune Response Evaluation Criteria in Solid Tumors.

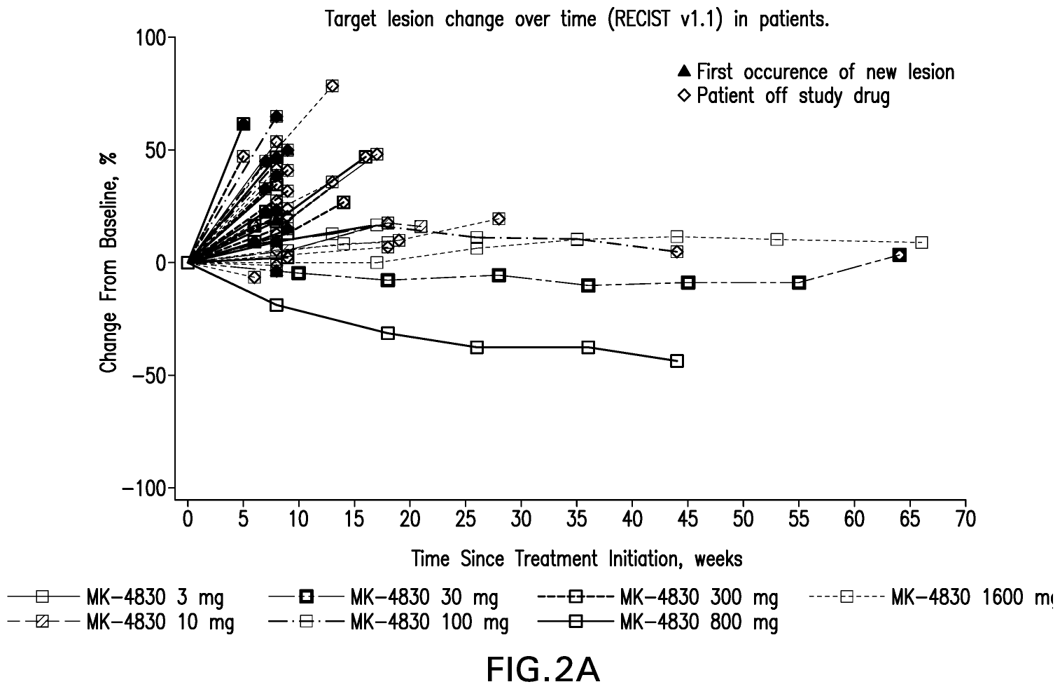

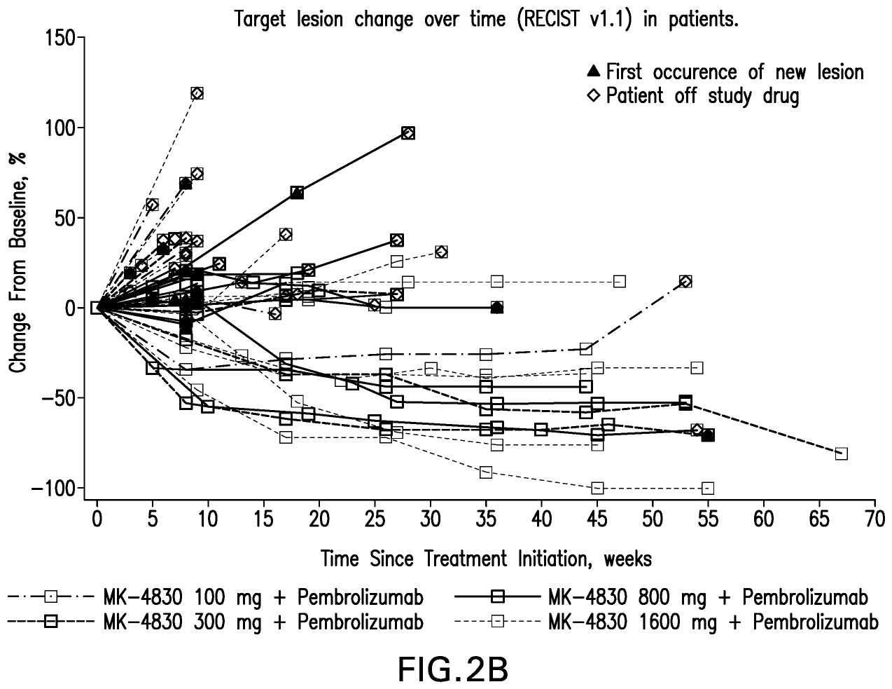

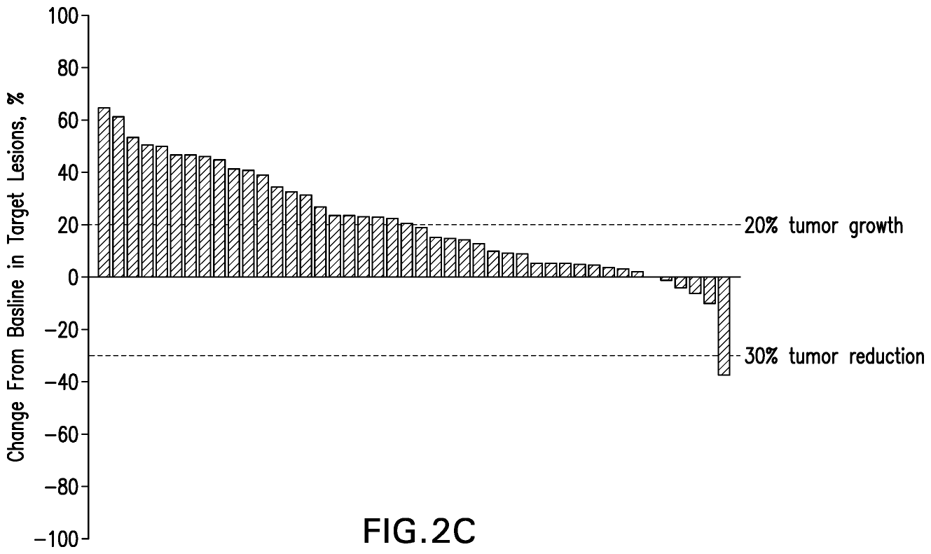

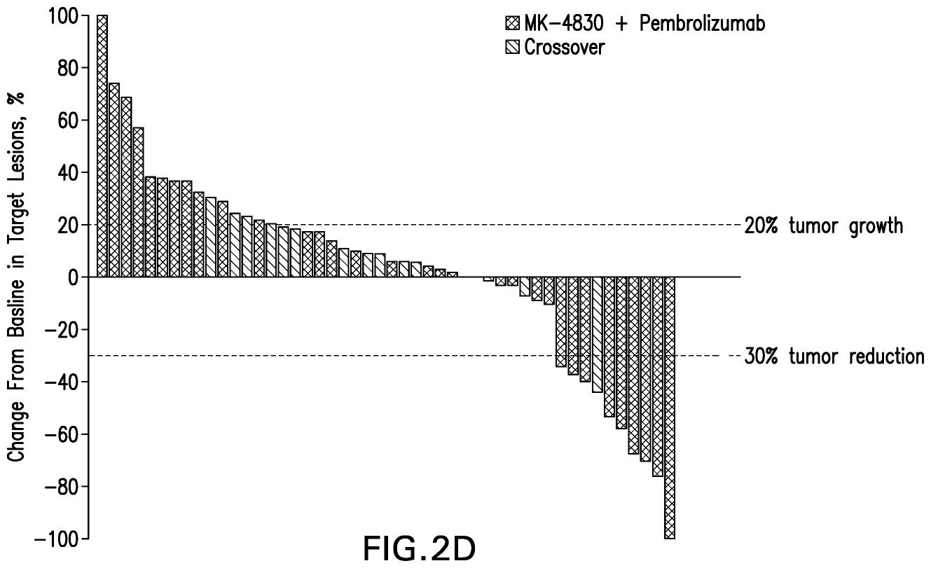

Eight of 34 patients receiving combination therapy of Antibody A and pembrolizumab achieved a confirmed response (complete response [CR], n=1; PR, n=7) and 6 additional patients had SD≥6 months (per Response Evaluation Criteria in Solid Tumors version 1.1 [RECIST v1.1] by investigator review) for an ORR of 24% and a disease control rate (DCR) of 41% (Table 5). One additional patient in the combination treatment group achieved an unconfirmed PR. In the crossover group, 1 patient with MSI-high colorectal cancer achieved a confirmed PR. Ten patients with a variety of recurrent advanced solid tumors (head and neck squamous cell carcinoma [HNSCC], n=2; gastric cancer, n=2; colorectal cancer, n=2; non-small cell lung cancer [NSCLC], n=1; dendritic sarcoma, n=1; papillary thyroid, n=1; Merkel cell carcinoma, n=1) had continued PRs at doses of Antibody A 100 mg (n=1), 300 mg (n=1), 800 mg (n=4), and 1600 mg (n=4). A total of 51% of patients with responses previously received≥3 lines of therapy. In addition, 5 of 11 patients (45%) whose disease had previously progressed on an anti-PD-1/PD-L1 alone or in combination with other agents achieved PR with Antibody A plus pembrolizumab. All responses were maintained for ≥6 months (FIGS. 2A and 2B). Reductions in target lesion size of ≥30% were observed in the patient receiving monotherapy and the 10 patients receiving combination therapy (including those who crossed over) (FIGS. 2C and 2D).

Biomarker Assessments—PD-L1, TMB, Tcell_{inf}GEP, and myeloid-specific biomarkers were assessed using archival or newly obtained tumor samples from all patients in the combination group of the study.

PD-L1 protein expression was assessed by immunohistochemistry using PD-L1 22C3 IHC pharmDx (Agilent, Carpinteria, CA) performed at Interpace Pharma Solutions.

TMB was assessed by DNA extracted from formalin-fixed paraffin-embedded (FFPE) tissue using the Qiagen FFPE kit. DNA fragmentation was performed using the Covaris® (Woburn, MA) M220 focused-ultrasonicator with the settings of 75W for peak incident power, 25% as duty factor, 1000 for cycles per burst, and a treatment time of 6 minutes per sample at 40° C. For the TruSight Oncology 500 (TS0500) next-generation sequencing (NGS) library preparation, 40 mg to 50 mg FFPE DNA per sample was used. Library preparation was performed manually in accordance with the manufacturer's protocol, with 24 samples per batch. NGS was performed on a NextSeq™ 550 (Illumina, San Diego, CA) instrument with 8 libraries per sequencing run. TS0500-TMB was reported using the TS0500 Local App version 1 (Illumina). The manufacturer's quality control criteria were used to determine whether a result was valid, including NGS library concentration of ≥1 ng/μL, median insert size of ≥70 base pairs, median exon coverage of ≥50 count, and percent of exons with coverage≥50 count of ≥90%.

Tcell_{inf}GEP and the mMDSC signature were assessed by RNA extracted from FFPE pretreatment tumor samples using the High Pure FFPE RNA Isolation Kit (Roche, Basel, Switzerland) according to the manufacturer's protocol. RNA sequencing was used to measure the 18-gene TcellinrGEP and the 218 genes of the mMDSC signature. Input sample data were generated using the HiSeq 4000 and the TruSeq RNA access library preparation method (Illumina). Gene-level fragments per kilobase million (FPKM) values were generated in OmicSoft Array Studio v11.0 (Qiagen, Hilden, Germany) according to the established RNA-sequencing pipeline. OmicSoft sequence aligner (OSA) as alignment algorithm, Human.B37.3 as alignment reference, and Ensembl.R75 as gene model were used. FPKM values were then converted to log₁₀(0.01+FPKM), and each sample was normalized by subtracting the 75th percentile evaluated over probes annotated as protein coding and assigned to chromosomes 1:22, X, and Y.

Tcell_{inf}GEP score was calculated according to the published formula as weighted sum over 18 genes using normalized values for the 18 genes and previously published 18-gene coefficients as weights (Ayers M, Lunceford J, Nebozhyn M, Murphy E, Loboda A, Kaufman D R, et al. IFN-gamma-related mRNA profile predicts clinical response to PD-1 blockade. J Clin Invest 2017; 127:2930-40). Note that the Tcell_{inf}GEP is calculated as a weighted average of its member genes, whereas the mMDSC signature score is a simple average. Expression of myeloid-specific biomarkers, mMDSC signature score, LILRB2 levels, and HLA-G were also evaluated. As previously described (Cristescu R, Nebozhyn M, Zhang C, Albright A, Kobie J, Huang L, et al. Pan-tumor analysis of the association of cancer and immune biology-related gene expression signatures with response to pembrolizumab monotherapy. ImmunoTher Cancer 2019; 7:282. P324), mMDSC signature scores are analyzed by adjusting the data for the Tcell_{inf}GEP relative to the clinical outcome to ensure the score is independent of the Tcell_{inf}GEP, which is in line with findings for the mMDSC signature score as a negatively associated factor in joint regression models with the Tcell_{inf}GEP (Cristescu R, Nebozhyn M, Zhang C, Albright A, Kobie J, Huang L, et al. Pan-tumor analysis of the association of cancer and immune biology-related gene expression signatures with response to pembrolizumab monotherapy. ImmunoTher Cancer 2019; 7:282. P324).

When evaluating patterns of clinical response in the present study, parallel use of the mMDSC signature score was analyzed by adjusting for the Tcell_{inf}GEP by simple linear regression of the expression levels of mMDSC signature scores on the Tcell_{inf}GEP and calculating the residual levels of mMDSC; this evaluates whether these putatively suppressive myeloid-related factors are higher or lower than expectations based on Tcell_{inf}GEP. The same approach was used for LILRB2 and HLA-G when comparing levels for responders and non-responders.

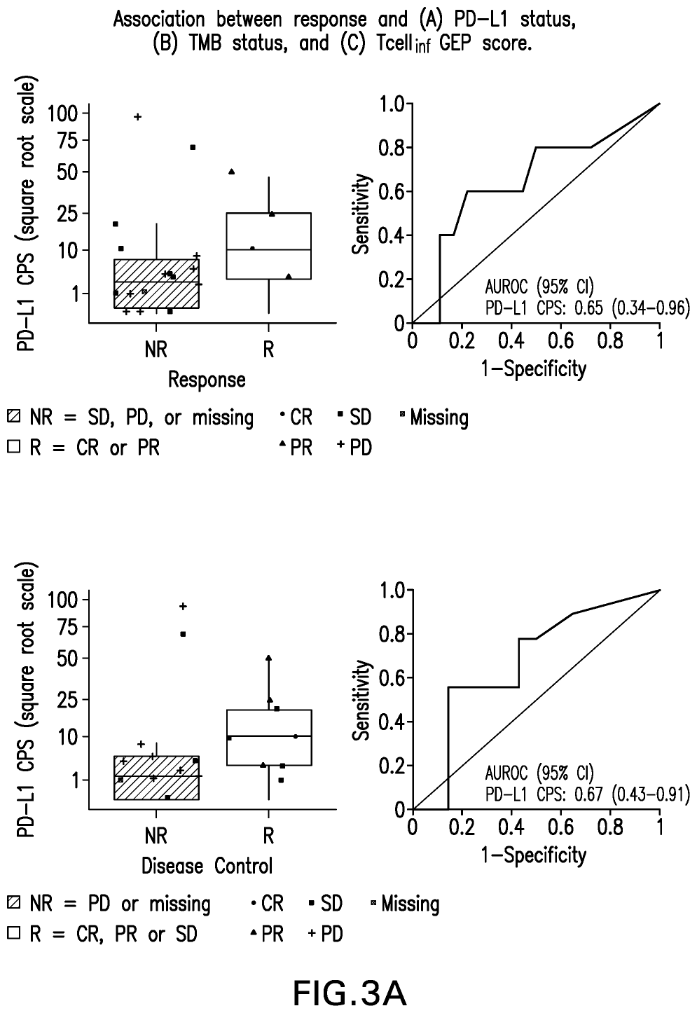

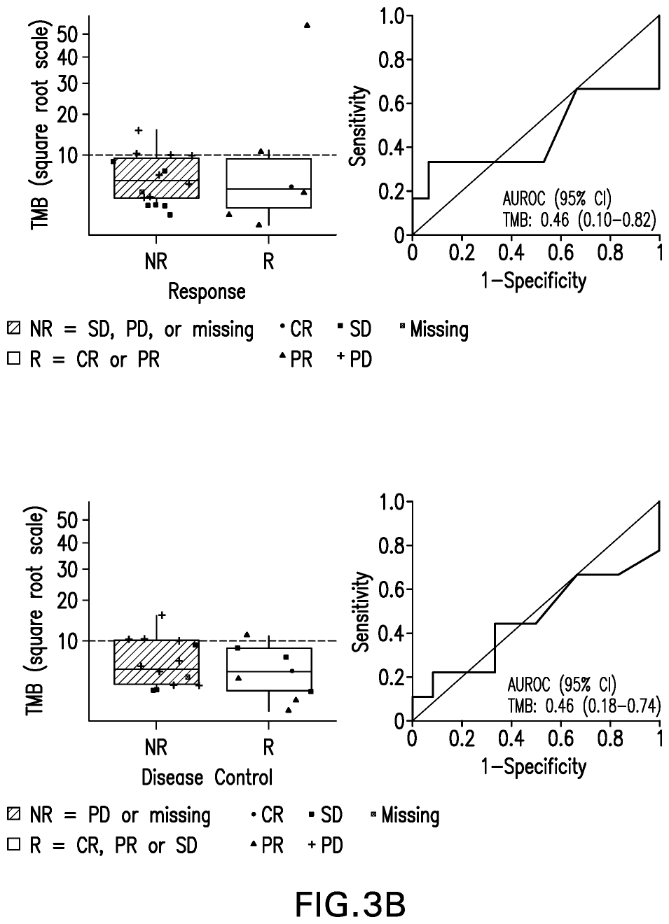

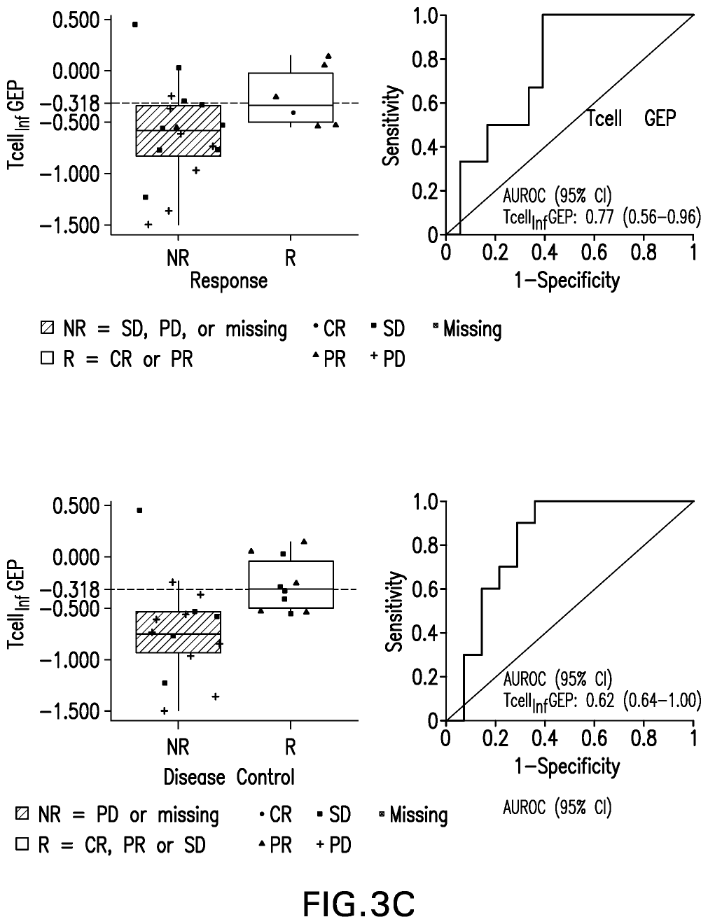

PD-L1 Biomarker—Of the 34 patients in the dose-escalation phase who received combination therapy, 25 (6 responders [CR or PR], 19 non-responders [SD, PD, or missing, including 5 patients with SD and progression-free survival (PFS) of ≥6 months]) had evaluable PD-L1 status (FIG. 3A). A general trend toward higher tumor PD-L1 combined positive score (CPS) expression was observed in responders.

TMB Biomarker—Twenty-one patients (6 responders [CR or PR], 15 non-responders [SD, PD, or missing, including 5 patients with SD and PFS of ≥6 months]) had evaluable TMB status. No trend toward higher TMB status was observed in patients who responded to study treatment (FIG. 3B).

Tcell_{inf}GEP Biomarker—Twenty-four patients (6 responders [CR or PR], 18 non-responders [SD, PD, or missing, including 4 patients with SD and PFS of ≥6 months]) had evaluable Tcell_{inf}GEP scores. The distribution of the Tcell_{inf}GEP score was higher in responders than in non-responders (FIG. 3C).

Statistical Analysis—Safety was evaluated in all patients who received≥1 dose of study treatment (all-patients-as-treated population), with patients grouped according to the treatment received. The dose limiting toxicity (DLT)-evaluable population comprised patients who experienced a DLT during the first cycle and those who completed the first cycle of treatment without a DLT.

Prespecified DLTs included the following: (1) grade 4 nonhematological toxicity (nonlaboratory); (2) grade 4 hematological toxicity lasting≥7 days: grade 4 thrombocytopenia of any duration and/or grade 3 thrombocytopenia associated with clinically significant bleeding; (3) any nonhematological AE grade≥3 in severity (exceptions: grade 3 fatigue lasting≤3 days; grade 3 diarrhea, nausea, or vomiting without use of antiemetics or antidiarrheals per standard of care; and grade 3 rash without the use of corticosteroids or anti-inflammatory agents per standard of care); (4) any grade 3 or grade 4 ALT, AST, or bilirubin laboratory values (exception: if AST or ALT was grade 2 at baseline [as in patients with liver metastases], then a DLT was defined as >2× above baseline); (5) any other nonhematological laboratory value if clinically significant medical intervention was required to treat the patient, or if the abnormality led to hospitalization, persisted for >1 week, or resulted in a drug-induced liver injury (exceptions: clinically nonsignificant, treatable, or reversible laboratory abnormality including alkaline phosphatase, gamma glutamyl transferase, or uric acid); (6) febrile neutropenia grade 3 or grade 4; (7) prolonged delay (>2 weeks) in initiation of cycle 2 because of treatment-related toxicity; and (8) any treatment-related toxicity that caused the patient to discontinue treatment during cycle 1. Peripheral blood Antibody A RO pharmacokinetics and ADAs were summarized by planned visit and time for each dose. ORR and DCR were assessed in the full analysis set, which included patients with measurable disease at baseline who received≥1 dose of study treatment. The clinical data cutoff date was Jul. 10, 2020.

### SEQUENCE LISTING

Table 9 below summarizes Antibody A sequences disclosed herein.

TABLE 9
SEQ ID NOS and Corresponding Sequences for Antibody A

| SEQ ID NO | Description | Sequence |
|---|---|---|
| 1 | Antibody A, | TGSSSNIGAGYDVH |
| | VL-CDR1 | |
| 2 | Antibody A, | GDSNRPS |
| | VL-CDR2 | |
| 3 | Antibody A, | QSFDNSLSAYV |
| | VL-CDR3 | |
| 4 | Antibody A, | ESVLTQPPSVSGAPGQRVTISCTGSSSNIGAGYDVHWYQQL |
| | VL | PGTAPKLLIYGDSNRPSGVPDRFSVSKSGASASLAITGLQA |
| | | EDEADYYCQSFDNSLSAYVFGGGTQLTVL |
| 5 | Antibody A, | ESVLTQPPSVSGAPGQRVTISCTGSSSNIGAGYDVHWYQQL |
| | light chain | PGTAPKLLIYGDSNRPSGVPDRFSVSKSGASASLAITGLQA |
| | | EDEADYYCQSFDNSLSAYVFGGGTQLTVLGQPKAAPSVTLF |
| | | PPSSEELQANKATLVCLISDFYPGAVTVAWKADSSPVKAGV |
| | | ETTTPSKQSNNKYAASSYLSLTPEQWKSHRSYSCQVTHEGS |
| | | TVEKTVAPTECS |
| 6 | Antibody A, | GYYWS |
| | VH-CDR1 | |
| 7 | Antibody A, | EINHAGSTNYNPSLKS |
| | VH-CDR2 | |
| 8 | Antibody A, | LPTRWVTTRYFDL |
| | VH-CDR3 | |
| 9 | Antibody A, | EVQLQQWGAGLLKPSETLSLTCAVYGGSFSGYYWSWIRQPP |
| | VH | GKGLEWIGEINHAGSTNYNPSLKSRVTISVDTSKNQFSLKL |
| | | SSVTAADTAVYYCARLPTRWVTTRYFDLWGRGTLVTVSS |
| 10 | Antibody A, | EVQLQQWGAGLLKPSETLSLTCAVYGGSFSGYYWSWIRQPP |
| | heavy chain | GKGLEWIGEINHAGSTNYNPSLKSRVTISVDTSKNQFSLKL |
| | | SSVTAADTAVYYCARLPTRWVTTRYFDLWGRGTLVTVSSAS |
| | | TKGPSVFPLAPCSRSTSESTAALGCLVKDYFPEPVTVSWNS |
| | | GALTSGVHTFPAVLQSSGLYSLSSVVTVPSSSLGTKTYTCN |
| | | VDHKPSNTKVDKRVESKYGPPCPPCPAPEFLGGPSVFLFPP |
| | | KPKDTLMISRTPEVTCVVVDVSQEDPEVQFNWYVDGVEVHN |
| | | AKTKPREEQFNSTYRVVSVLTVLHQDWLNGKEYKCKVSNKG |
| | | LPSSIEKTISKAKGQPREPQVYTLPPSQEEMTKNQVSLTCL |
| | | VKGFYPSDIAVEWESNGQPENNYKTTPPVLDSDGSFFLYSR |
| | | LTVDKSRWQEGNVFSCSVMHEALHNHYTQKSLSLSLGK |

While the present invention is described herein with reference to illustrated embodiments, it should be understood that the invention is not limited hereto. Those having ordinary skill in the art and access to the teachings herein will recognize additional modifications and embodiments within the scope thereof.

1. A method for determining the eligibility of a human subject having a malignancy for treatment with an anti-ILT4 and anti-PD-1 combination therapy comprising:
determining the number of viable PD-L1 positive tumor cells, the number of viable PD-L1 negative tumor cells, and the number of viable PD-L1 positive mononuclear inflammatory cells (MIC) in a tumor tissue sample from the subject having a malignancy; and
calculating a combined positive score (CPS) for the tumor tissue sample using the formula:
$$CPS = \frac{PD - L 1 positive tumor cells + PD - L 1 positive MIC}{PD - L 1 positive tumor cells + PD - L 1 negative tumor cells} \times 100 ;$$
wherein the subject is eligible for treatment with the anti-ILT4 and anti-PD-1 combination therapy when the CPS is equal to or greater than a threshold value.

2-4. (canceled)

5. The method of claim 1, wherein the tumor tissue sample is a tissue section of a tumor biopsy.

6. The method of claim 5, wherein PD-L1 is detected by immunohistochemistry (IHC) staining.

7. The method of claim 5, wherein the tumor tissue section is a formalin fixed and embedded in paraffin wax (FFPE) tumor tissue section.

8-9. (canceled)

10. The method of claim 5, wherein the viable PD-L1 positive tumor cells, the number of viable PD-L1 negative tumor cells, and the number of viable PD-L1 positive MIC are counted in the tumor nests and the adjacent supporting stroma of the tumor tissue sample.

11. (canceled)

12. The method of claim 1, wherein the method further comprises, prior to the determining step, contacting the tumor tissue sample with an anti-ILT4 antibody or a binding fragment thereof and an anti-PD-1 antibody or a binding fragment thereof.

13. The method of claim 12, wherein the anti-ILT4 antibody or a fragment thereof comprises a VL-CDR1 of SEQ ID No. 1, a VL-CDR2 of SEQ ID No. 2, and a VL-CDR3 of SEQ ID No. 3; and a VH-CDR1 of SEQ ID No. 6, a VH-CDR2 of SEQ ID No. 7, and a VH-CDR3 of SEQ ID No. 8.

14. The method of claim 12, wherein the anti-ILT4 antibody or a binding fragment thereof comprises a light chain sequence of SEQ ID NO. 5 and a heavy chain sequence of SEQ ID NO. 10.

15. The method of claim 12, wherein the anti-PD-1 antibody or a binding fragment thereof is selected from the group consisting of: dostarlimab, nivolumab, pembrolizumab, BMS-936559, MPDL3280A, cemiplimab, and MEDI4736.

16. The method of claim 12, wherein the anti-ILT4 antibody or a binding fragment thereof comprises a light chain sequence of SEQ ID NO. 5 and a heavy chain sequence of SEQ ID NO. 10; and wherein the anti-PD-1 antibody or a binding fragment thereof is pembrolizumab.

17. A method for determining the eligibility of a human subject having a malignancy for treatment with an anti-ILT4 and anti-PD-1 combination therapy comprising determining the T-cell-inflamed gene expression profile (Tcell_{inf}GEP) score from the weighted average of its member genes; wherein the subject is eligible for treatment with the anti-ILT4 and anti-PD-1 combination therapy when the Tcell_{inf}GEP score is equal to or greater than a threshold value.

18-19. (canceled)

20. The method of claim 17, wherein the threshold value is equal to or greater than −0.5; and wherein the anti-ILT4 and anti-PD-1 combination therapy comprises a light chain sequence of SEQ ID NO. 5 and a heavy chain sequence of SEQ ID NO. 10 and pembrolizumab combination therapy.

21. The method of claim 1, wherein the malignancy is selected from the group consisting of: gastric cancer, head and neck cancer, renal cell carcinoma, urothelial carcinoma, ovarian carcinoma, myeloma, melanoma, lung cancer, squamous cell carcinoma, classical Hodgkin lymphoma, breast cancer, triple negative breast cancer, hormone receptor positive (ER and/or PR) and Her2 positive breast cancer, MSI high colorectal cancer, non-small cell lung cancer, small cell lung cancer, salivary gland carcinoma, vulvar carcinoma, thyroid carcinoma, anal canal carcinoma, biliary carcinoma, mesothelioma, cervical carcinoma, and neuroendocrine carcinoma.

22. A method for treating a human subject having a malignancy, the method comprising the steps of:
determining the number of viable PD-L1 positive tumor cells, the number of viable PD-L1 negative tumor cells, and the number of viable PD-L1 positive mononuclear inflammatory cells (MIC) in a tumor tissue sample from the subject; and
calculating a combined positive score (CPS) for the tumor tissue sample using the formula:
$$CPS = \frac{PD - L 1 positive tumor cells + PD - L 1 positive MIC}{PD - L 1 positive tumor cells + PD - L 1 negative tumor cells} \times 100 ;$$
wherein the subject is treated with the anti-ILT4 and anti-PD-1 combination therapy when the CPS is equal to or greater than a threshold value.

23-32. (canceled)

33. The method of claim 22, wherein the anti-ILT4 antibody or a binding fragment thereof comprises a light chain sequence of SEQ ID No. 5 and a heavy chain sequence of SEQ ID No. 10.

34. The method of claim 22, wherein the anti-PD-1 antibody or a binding fragment thereof is selected from the group consisting of: cemiplimab, nivolumab, pembrolizumab, BMS-936559, MPDL3280A, dostarlimab, and MEDI4736.

35. The method of claim 22, wherein the anti-ILT4 antibody or a fragment thereof comprises a VL-CDR1 of SEQ ID No. 1, a VL-CDR2 of SEQ ID No. 2, and a VL-CDR3 of SEQ ID No. 3; and a VH-CDR1 of SEQ ID No. 6, a VH-CDR2 of SEQ ID No. 7, and a VH-CDR3 of SEQ ID No. 8, and wherein the anti-PD-1 antibody or binding fragment thereof is pembrolizumab.

36. The method of claim 22, wherein the anti-ILT4 antibody or a binding fragment thereof comprises a light chain sequence of SEQ ID No. 5 and a heavy chain sequence of SEQ ID No. 10; and wherein the anti-PD-1 antibody or a binding fragment thereof is pembrolizumab.

37. (canceled)

38. A method for treating a human subject having a malignancy, the method comprising the steps of:
treating the subject with an anti-ILT4 antibody or a binding fragment thereof,
determining the number of viable PD-L1 positive tumor cells, the number of viable PD-L1 negative tumor cells, and the number of viable PD-L1 positive mononuclear inflammatory cells (MIC) in a tumor tissue sample from the subject who has been treated with the anti-ILT4 antibody or a binding fragment thereof, and
calculating a combined positive score (CPS) for the tumor tissue sample using the formula:
$$CPS = \frac{PD - L 1 positive tumor cells + PD - L 1 positive MIC}{PD - L 1 positive tumor cells + PD - L 1 negative tumor cells} \times 100 ;$$
wherein the subject is further treated with the anti-ILT4 and anti-PD-1 combination therapy when the CPS is equal to or greater than a threshold value.

39-50. (canceled)

51. The method of claim 38, wherein the anti-ILT4 antibody or a fragment thereof comprises a VL-CDR1 of SEQ ID No. 1, a VL-CDR2 of SEQ ID No. 2, and a VL-CDR3 of SEQ ID No. 3; and a VH-CDR1 of SEQ ID No. 6, a VH-CDR2 of SEQ ID No. 7, and a VH-CDR3 of SEQ ID No. 8, and wherein the anti-PD-1 antibody or a binding fragment thereof is pembrolizumab.

52. The method of claim 38, wherein the anti-ILT4 antibody or a binding fragment thereof comprises a light chain sequence of SEQ ID No. 5 and a heavy chain sequence of SEQ ID No. 10; and wherein the anti-PD-1 antibody or a binding fragment thereof is pembrolizumab.

53. (canceled)


SEQUENCE LISTING (10 sequences)

SEQ ID NO: 1 - protein, 14 aa, synthetic construct
TGSSSNIGAGYDVH

SEQ ID NO: 2 - protein, 7 aa, synthetic construct
GDSNRPS

SEQ ID NO: 3 - protein, 11 aa, synthetic construct
QSFDNSLSAYV

SEQ ID NO: 4 - protein, 111 aa, synthetic construct
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVL

SEQ ID NO: 5 - protein, 217 aa, synthetic construct
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 5 aa, synthetic construct
GYYWS

SEQ ID NO: 7 - protein, 16 aa, synthetic construct
EINHAGSTNYNPSLKS

SEQ ID NO: 8 - protein, 13 aa, synthetic construct
LPTRWVTTRYFDL

SEQ ID NO: 9 - protein, 121 aa, synthetic construct
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSS

SEQ ID NO: 10 - protein, 448 aa, synthetic construct
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK